(19) United States
(12) Patent Application Publication
(10) Pub. No.: US 2023/0002786 A1
(43) Pub. Date: Jan. 5, 2023

(54) SYNTHETIC ADENO-ASSOCIATED VIRUS INVERTED TERMINAL REPEATS AND METHODS OF THEIR USE AS PROMOTERS

(71) Applicant: The University of North Carolina at Chapel Hill, Chapel Hill, NC (US)

(72) Inventor: Richard Jude Samulski, Hillsborough, NC (US)

(21) Appl. No.: 17/791,303

(22) PCT Filed: Jan. 7, 2021
(86) PCT No.: PCT/US2021/012514

§ 371 Date: Jul. 7, 2022

Related U.S. Application Data
(60) Provisional application No. 62/957,882, filed on Jan. 7, 2020.

(51) Int. Cl. C12N 15/86; A61K 48/00

(52) U.S. Cl. CPC C12N 15/86; A61K 48/0066; C12N 2750/14123; C12N 2750/14122; C12N 2750/14143

(57) ABSTRACT

The present invention provides methods and compositions comprising an adeno-associated vims (AAV) synthetic inverted terminal repeat (ITR), wherein the ITR may have modified promoter transcriptional function. Additionally provided are vectors and virus particles comprising the same, as well as methods of their use.

### STATEMENT OF PRIORITY

This application claims the benefit, under 35 U.S.C. § 119(e), of U.S. Provisional Application No. 62/957,882, filed on Jan. 7, 2020, the entire contents of which are incorporated by reference herein.

### STATEMENT OF GOVERNMENT SUPPORT

This invention was made with government support under Grant Numbers RO1AI072176-06A and RO1AR064369-01A awarded by the National Institutes of Health. The government has certain rights in the invention.

### STATEMENT REGARDING ELECTRONIC FILING OF A SEQUENCE LISTING

A Sequence Listing in ASCII text format, submitted under 37 C.F.R. § 1.821, entitled 5470.875.WO ST25.txt, 3,464 bytes in size, generated on Jan. 7, 2021 and filed via EFS-Web, is provided in lieu of a paper copy. This Sequence Listing is hereby incorporated herein by reference into the specification for its disclosures.

### FIELD OF THE INVENTION

The present invention relates to synthetic inverted terminal repeats (ITRs) from adeno-associated virus (AAV), virus capsids and virus vectors comprising the same, as well as methods of their use.

### BACKGROUND OF THE INVENTION

Adeno-associated viral vectors have been used in laboratory and clinical settings for efficient gene delivery. In these vectors, 96% of the AAV genome is replaced with a gene cassette of interest, leaving only the 145 base-pair inverted terminal repeat sequences. These cis-elements primarily from AAV serotype 2 are required for genome rescue, replication, packaging, and vector persistence. The AAV2 ITR sequence has inherent transcriptional activity, which may confound intended transgene expression in therapeutic applications.

The present invention overcomes previous shortcomings in the art by providing synthetic inverted terminal repeats with modified promoter function, AAV vectors comprising these ITRs and methods for their use in gene therapy.

### SUMMARY OF THE INVENTION

A first aspect of the present invention provides a polynucleotide comprising at least one synthetic adeno-associated virus (AAV) inverted terminal repeat (ITR), wherein said ITR comprises: (a) an AAV rep binding element (RBE); (b) a B-loop; (c) a C-loop; (d) one or more nicking-stem loops; (e) a D-region; (f) an AAV terminal resolution sequence; and (g) an AAV RBE′ element; wherein (a)-(g) are from any AAV serotype that is not AAV2 or AAV3, and (i) wherein the RBE′ element comprises a non-complementary loop TTT sequence at a position that corresponds to nucleotide positions 73 to 75, wherein the nucleotide numbering is based on the nucleotide sequence of SEQ ID NO:1 (AAV2); (ii) wherein the B-loop comprises a nucleotide sequence that has 80% sequence identity to the nucleotide sequence of the B-loop of ITR2 or ITR3 (i.e., the ITR of AAV2 or AAV3, respectively) at a position that corresponds to nucleotide positions 43-61, wherein the nucleotide numbering is based on the nucleotide sequence of SEQ ID NO:1; (iii) wherein the C-loop comprises a nucleotide sequence that has 80% sequence identity to the nucleotide sequence of the C-loop of ITR2 or ITR3 at a position that corresponds to nucleotide positions 65-83, wherein the nucleotide numbering is based on the nucleotide sequence of SEQ ID NO:1; (iv) wherein the D-region comprises a nucleotide sequence that has 80% sequence identity to the nucleotide sequence of the D-region of ITR2 or ITR3 at a position that corresponds to nucleotide positions 125-145, wherein the nucleotide numbering is based on the nucleotide sequence of SEQ ID NO:1; and/or (v) wherein at least one of the one or more nicking-stem loops comprises a G substitution at a position that corresponds to nucleotide position 4 and/or a C substitution at a position that corresponds to nucleotide position 122 (e.g., C4G and/or G122C), wherein the nucleotide numbering is based on the nucleotide sequence of SEQ ID NO:1.

A second aspect of the present invention provides a polynucleotide comprising at least one synthetic adeno-associated virus (AAV) inverted terminal repeat (ITR), wherein said ITR comprises: (a) an AAV rep binding element (RBE); (b) a B-loop; (c) a C-loop; (d) one or more nicking-stem loops; (e) a D-region; (f) an AAV terminal resolution sequence; and (g) an AAV RBE′ element; wherein (a)-(g) are from any AAV serotype that is not AAV1 or AAV6, and (i) wherein the RBE′ element comprises a non-complementary loop TCT sequence at a position that corresponds to nucleotide positions 73 to 75, wherein the nucleotide numbering is based on the nucleotide sequence of SEQ ID NO:1; (ii) wherein the B-loop comprises a nucleotide sequence that has 80% sequence identity to the nucleotide sequence of the B-loop of ITR1 or ITR6 (i.e., the ITR of AAV1 or AAV6) at a position that corresponds to nucleotide positions 43-61, wherein the nucleotide numbering is based on the nucleotide sequence of SEQ ID NO:1; (iii) wherein the C-loop comprises a nucleotide sequence that has 80% sequence identity to the nucleotide sequence of the C-loop of ITR1 or ITR6 at a position that corresponds to nucleotide positions 65-83, wherein the nucleotide numbering is based on the nucleotide sequence of SEQ ID NO:1; (iv) wherein the D-region comprises a nucleotide sequence that has 80% sequence identity to the nucleotide sequence of the D-region of ITR1 or ITR6 at a position that corresponds to nucleotide positions 125-145, wherein the nucleotide numbering is based on the nucleotide sequence of SEQ ID NO:1; and/or (v) wherein at least one of the one or more nicking-stem loops comprises a C substitution at a position that corresponds to nucleotide position 4 and/or a G substitution at a position that corresponds to nucleotide position 122 (e.g., G4C and/or C122G), wherein the nucleotide numbering is based on the nucleotide sequence of SEQ ID NO:1.

Additional aspects of the invention relate to vectors, recombinant AAV particles, and chimeric AAV particles comprising a polynucleotide of the present invention.

Another aspect of the present invention provides a method of transcribing a heterologous nucleotide sequence in a cell, comprising introducing into the cell a polynucleotide of the present invention.

Another aspect of the present invention provides a method of delivering a nucleic acid to a cell, comprising introducing into a cell a recombinant AAV particle of the present invention.

An additional aspect of the present invention provides a method of producing a recombinant AAV particle, comprising providing to a cell permissive for AAV replication: (a) a recombinant AAV template comprising (i) a heterologous nucleic acid, and (ii) a synthetic ITR of the present invention; and (b) a polynucleotide comprising Rep coding sequences and Cap coding sequences; under conditions sufficient for the replication and packaging of the recombinant AAV template; whereby recombinant AAV particles are produced in the cell.

An additional aspect of the present invention provides a method of producing a recombinant AAV particle, comprising providing to a cell permissive for AAV replication: (a) a recombinant AAV template comprising (i) a heterologous nucleic acid, and (ii) a wildtype ITR from any AAV serotype or a synthetic ITR of the present invention; and (b) a polynucleotide comprising Rep coding sequences and Cap coding sequences, wherein the Rep and Cap coding sequences are from a different AAV serotype; under conditions sufficient for the replication and packaging of the recombinant AAV template; whereby recombinant AAV particles are produced in the cell.

Another aspect of the present invention provides a method of administering a nucleic acid to a mammalian subject comprising administering to the mammalian subject a cell that has been contacted with a recombinant AAV particle of the present invention under conditions sufficient for the AAV particle vector genome to enter the cell.

Another aspect of the present invention provides a method of administering a nucleic acid to a mammalian subject comprising administering to the mammalian subject a recombinant AAV particle of the present invention.

A further aspect of the present invention provides a method of enhancing promoter function of an adeno-associated virus (AAV) inverted terminal repeat (ITR) relative to a wildtype (e.g., unmodified) ITR, wherein said ITR comprises: (a) an AAV rep binding element (RBE); (b) a B-loop; (c) a C-loop; (d) one or more nicking-stem loops; (e) a D-region; (f) an AAV terminal resolution sequence; and (g) an AAV RBE′ element; wherein (a)-(g) are from any AAV serotype that is not AAV2 or AAV3, comprising substituting one or more of the following: (i) a non-complementary loop TTT sequence at a position that corresponds to nucleotide positions 73 to 75, wherein the nucleotide numbering is based on the nucleotide sequence of SEQ ID NO:1; (ii) a nucleotide sequence that has 80% sequence identity to the nucleotide sequence of the B-loop of ITR2 or ITR3 (i.e., the ITR of AAV2 or AAV3, respectively) at a position that corresponds to nucleotide positions 43-61, wherein the nucleotide numbering is based on the nucleotide sequence of SEQ ID NO:1; (iii) a nucleotide sequence that has 80% sequence identity to the nucleotide sequence of the C-loop of ITR2 or ITR3 at nucleotide positions 65-83, wherein the nucleotide numbering is based on the nucleotide sequence of SEQ ID NO:1; (iv) a nucleotide sequence that has 80% sequence identity to the nucleotide sequence of the D-region of ITR2 or ITR3 at nucleotide positions 125-145, wherein the nucleotide numbering is based on the nucleotide sequence of SEQ ID NO:1; and/or (v) a G substitution at nucleotide position 4 and/or a C substitution at nucleotide position 122 (e.g., C4G and/or G122C), wherein the nucleotide numbering is based on the nucleotide sequence of SEQ ID NO:1.

A further aspect of the present invention provides a method of reducing promoter function of an adeno-associated virus (AAV) inverted terminal repeat (ITR) relative to a wildtype (e.g., unmodified) ITR, wherein said ITR comprises: (a) an AAV rep binding element (RBE); (b) a B-loop; (c) a C-loop; (d) one or more nicking-stem loops; (e) a D-region; (f) an AAV terminal resolution sequence; and (g) an AAV RBE′ element; wherein (a)-(g) are from any AAV serotype that is not AAV1 or AAV6, comprising substituting one or more of the following: (i) a non-complementary loop TCT sequence at a position that corresponds to nucleotide positions 73 to 75, wherein the nucleotide numbering is based on the nucleotide sequence of SEQ ID NO:1; (ii) a nucleotide sequence that has 80% sequence identity to the nucleotide sequence of the B-loop of ITR1 or ITR6 (i.e., the ITR of AAV1 or AAV6) at nucleotide positions 43-61, wherein the nucleotide numbering is based on the nucleotide sequence of SEQ ID NO:1; (iii) a nucleotide sequence that has 80% sequence identity to the nucleotide sequence of the C-loop of ITR1 or ITR6 at nucleotide positions 65-83, wherein the nucleotide numbering is based on the nucleotide sequence of SEQ ID NO:1; (iv) a nucleotide sequence that has 80% sequence identity to the nucleotide sequence of the D-region of ITR1 or ITR6 at nucleotide positions 125-145, wherein the nucleotide numbering is based on the nucleotide sequence of SEQ ID NO:1; and/or (v) a C substitution at nucleotide position 4 and/or a G substitution at nucleotide position 122 (e.g., G4C and/or C122G), wherein the nucleotide numbering is based on the nucleotide sequence of SEQ ID NO:1.

These and other aspects of the invention are addressed in more detail in the description of the invention set forth below.

### BRIEF DESCRIPTION OF THE DRAWINGS

FIGS. 1A-1C show diagrams of the sequences and structures of AAV ITRs from serotypes 1-4, 6 and 7. FIG. 1A shows a diagram of the AAV2 ITR (“ITR2”) (SEQ ID NO:1) with the Rep binding element (RBE) and RBE′ in bold. The terminal resolution nicking site TT dinucleotide is in red. FIG. 1B shows a diagram of a consensus ITR sequence (SEQ ID NO:7). Locations of nucleotide differences between ITR sequences 1-4, 6-7 are highlighted in red. The red nucleotides are in IUPAC code where Y is C or T, R is A or G, S is G or C, W is A or T, K is G or T, M is A or C, B is G or T or C, V is G or C or A, and N is any nucleotide. Colored outlines denote the A (black), B (blue), C (grey), and D (green) regions in the ITR. FIG. 1C shows diagrams of the sequences and structures of ITRs 1 (SEQ ID NO:2), 3 (SEQ ID NO:3), 4 (SEQ ID NO:4), 6 (SEQ ID NO:5), and 7 (SEQ ID NO:6). Bolded letters denote non-conserved nucleotides between the ITR sequences.

FIGS. 2A-2G show graphs of luciferase activity from cell lines infected with AAV(1-4, 6-7)/2-ITR-luciferase vectors. FIG. 2A shows a graph of HEK293 cells infected with AAV2/2-ITR-luciferase or AAV2/2-CBA-luciferase at 1E5 vg/cell. 2 days post infection, the cells were lysed and luciferase activity was measured using luciferin substrate. Values shown are raw RLUs. FIGS. 2B-2D show graphs of the indicated cell lines infected with AAV1/2-ITR-luciferase, AAV2/2-ITR-luciferase, or AAV7/2-ITR-luciferase at 2E5 vg/cell. 2 days post infection, the cells were lysed and luciferase activity was measured using luciferin substrate. RLU values were normalized to total cellular protein added to the luciferase assay, as measured by BCA, then all values were normalized to ITR2. Each AAVN/2-ITR-luciferase was made in triplicate batches, titered together, and then cells were infected in triplicate. FIGS. 2E-2F show graphs of the indicated cell lines infected with AAV1/2-ITR-luciferase, AAV2/2-ITR-luciferase, AAV3/2-ITR-luciferase, AAV4/2-ITR-luciferase, or AAV6/2-ITR-luciferase at 2E5 vg/cell. 2 days post infection, the cells were lysed and luciferase activity was measured using luciferin substrate. RLU values were normalized to total cellular protein added to the luciferase assay, as measured by BCA, then all values were normalized to ITR2. Each AAVN/2-ITR-luciferase was made in triplicate batches, titered together, and then cells were infected in triplicate. p values are indicated as *<0.0001, #<0.001, and \hat{ }<0.01.

FIG. 3 shows a graph of luciferase activity from HEK293 cells infected with AAV1/1 and AAV2/1-ITR-luciferase vectors. HEK293 cells were infected in triplicate with three biological replicates of AAV1/1, AAV2/1, AAV1/2, or AAV2/2-ITR-luciferase vectors at 2E5 vg/cell. 2 days post infection, the cells were lysed and luciferase activity was measured using luciferin substrate. RLU values were normalized to total cellular protein added to the luciferase assay, as measured by BCA, then all values were normalized to ITR2 values within each capsid group (i.e., AAV1/1 and AAV2/1 were normalized to AAV2/1. AAV1/2 and AAV2/2 were normalized to AAV2/2). There is no statistical difference between AAV1/1 v AAV1/2.

FIG. 4A shows a diagram of transcription start sites (TSS) from ITR1-4, 6, or 7 (SEQ ID NOs:1-6) that promoted luciferase mRNA. HEK293 cells were infected with AAV(1-4, 6, or 7)/2-ITR-luciferase at 2E5 vg/cell and total RNA was harvested 3 days post infection. RNA was reverse transcribed using luciferase specific primers. Final cDNA products were analyzed by NGS. Black arrows indicate a TSS with 1% or higher of read sequences. The RBE′ and RBE are indicated in bold. The trs is denoted in red.

FIG. 4B shows a diagram indicating putative transcription factor binding sites in ITR2 (SEQ ID NO:1).

FIG. 4C shows a schematic identifying transcription start sites from ITR2 (SEQ ID NO:1) or ITR7 (SEQ ID NO:6) promoted mRNA. HEK293 cells were infected with AAV2/2 (top) or AAV7/2-ITR-luciferase (bottom) and total RNA was harvested 3 days post infection. RNA was reverse transcribed using luciferase specific primers. Final cDNA products were analyzed by next generation sequencing. Arrows indicate the TSS, the number above the arrow indicating the number of transcripts that could be traced back to that site.

FIGS. 5A-5B show images of luciferase assays. Luciferase activity from mice injected with AAV(1-4, 6)/9-ITR-cre recombinase 4-6 weeks old male FVB.129S6(B6)-Gt(ROSA)26Sor^{tm1(Luc)Kael}/J mice were injected with 100 μl of 1E9 vg of AAV(1-4, 6)/9-ITR-cre recombinase. At 3 weeks (FIG. 5A) and at 9 weeks (FIG. 5B) post AAV injection, mice were given 100 μl of luciferase substrate i.p. and photons were recorded.

### DETAILED DESCRIPTION OF THE INVENTION

The present invention will now be described with reference to the accompanying drawings, in which representative embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, and other references mentioned herein are incorporated by reference herein in their entirety.

### Definitions

The singular forms “a,” “an” and “the” are intended to include the plural forms as well, unless the context clearly indicates otherwise.

Furthermore, the term “about,” as used herein when referring to a measurable value such as an amount of the length of a polynucleotide or polypeptide sequence, dose, time, temperature, and the like, is meant to encompass variations of ±20%, ±10%, ±5%, ±1%, ±0.5%, or even ±0.1% of the specified amount.

Also as used herein, “and/or” refers to and encompasses any and all possible combinations of one or more of the associated listed items, as well as the lack of combinations when interpreted in the alternative (“or”).

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

To illustrate further, if, for example, the specification indicates that a particular amino acid can be selected from A, G, I, L and/or V, this language also indicates that the amino acid can be selected from any subset of these amino acid(s) for example A, G, I or L; A, G, I or V; A or G; only L; etc., as if each such subcombination is expressly set forth herein. Moreover, such language also indicates that one or more of the specified amino acids can be disclaimed. For example, in particular embodiments the amino acid is not A, G or I; is not A; is not G or V; etc., as if each such possible disclaimer is expressly set forth herein.

As used herein, the terms “reduce,” “reduces,” “reduction” and similar terms mean a decrease of at least about 5%, 10%, 15%, 20%, 25%, 35%, 50%, 75%, 80%, 85%, 90%, 95%, 97%, 98%, 99%, 100% or more.

As used herein, the terms “enhance,” “enhances,” “enhancement” and similar terms indicate an increase of at least about 10%, 20%, 25%, 50%, 75%, 100%, 150%, 200%, 300%, 400%, 500% or more.

As used herein, the term “polypeptide” encompasses both peptides and proteins, unless indicated otherwise.

A “polynucleotide,” “nucleic acid,” or “nucleic acid molecule” as used herein is a sequence of nucleotide bases, and may be RNA, DNA or DNA-RNA hybrid sequences (including both naturally occurring and non-naturally occurring nucleotide), but in representative embodiments are either single or double stranded DNA sequences.

As used herein, an “isolated” polynucleotide (e.g., an “isolated DNA” or an “isolated RNA”) means a polynucleotide at least partially separated from at least some of the other components of the naturally occurring organism or virus, for example, the cell or viral structural components or other polypeptides or nucleic acids commonly found associated with the polynucleotide. In representative embodiments an “isolated” nucleotide is enriched by at least about 10-fold, 100-fold, 1000-fold, 10,000-fold or more as compared with the starting material.

Likewise, an “isolated” polypeptide means a polypeptide that is at least partially separated from at least some of the other components of the naturally occurring organism or virus, for example, the cell or viral structural components or other polypeptides or nucleic acids commonly found associated with the polypeptide. In representative embodiments an “isolated” polypeptide is enriched by at least about 10-fold, 100-fold, 1000-fold, 10,000-fold or more as compared with the starting material.

As used herein, by “isolate” or “purify” (or grammatical equivalents) a virus vector, it is meant that the virus vector is at least partially separated from at least some of the other components in the starting material. In representative embodiments an “isolated” or “purified” virus vector is enriched by at least about 10-fold, 100-fold, 1000-fold, 10,000-fold or more as compared with the starting material.

As used herein, the term “chimera,” “chimeric,” and/or “fusion protein” refer to an amino acid sequence (e.g., polypeptide) generated non-naturally by deliberate human design comprising, among other components, an amino acid sequence of a protein of interest and/or a modified variant and/or active fragment thereof (a “backbone”), wherein the protein of interest comprises modifications (e.g., substitutions such as singular residues and/or contiguous regions of amino acid residues) from different wild type reference sequences (chimera), optionally linked to other amino acid segments (fusion protein). The different components of the designed protein may provide differing and/or combinatorial function. Structural and functional components of the designed protein may be incorporated from differing and/or a plurality of source material. The designed protein may be delivered exogenously to a subject, wherein it would be exogenous in comparison to a corresponding endogenous protein.

A “therapeutic polypeptide” is a polypeptide or peptide that can alleviate, reduce, prevent, delay and/or stabilize symptoms that result from an absence or defect in a protein in a cell or subject and/or is a polypeptide that otherwise confers a benefit to a subject.

By the terms “treat,” “treating” or “treatment of” (and grammatical variations thereof) it is meant that the severity of the subject's condition is reduced, at least partially improved or stabilized and/or that some alleviation, mitigation, decrease or stabilization in at least one clinical symptom is achieved and/or there is a delay in the progression of the disease or disorder.

The terms “prevent,” “preventing” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset is less than what would occur in the absence of the present invention.

A “treatment effective,” “therapeutic,” or “effective” amount as used herein is an amount that is sufficient to provide some improvement or benefit to the subject. Alternatively stated, a “treatment effective,” “therapeutic,” or “effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

The terms “heterologous nucleotide sequence,” “heterologous nucleic acid,” or “heterologous nucleic acid molecule” are used interchangeably herein and refer to a sequence that is not naturally occurring in the virus. Generally, the heterologous nucleic acid comprises an open reading frame that encodes a polypeptide or nontranslated RNA of interest (e.g., for delivery to a cell or subject).

As used herein, the terms “virus vector,” “vector” or “gene delivery vector” refer to a virus (e.g., AAV) particle that functions as a nucleic acid delivery vehicle, and which comprises the vector genome (e.g., viral DNA [vDNA]) packaged within a virion.

Alternatively, in some contexts, the term “vector” may be used to refer to the vector genome/vDNA alone.

As used herein when referring to viruses, the terms “vector,” “particle,” and “virion” may be used interchangeably.

The virus vectors of the invention can further be duplexed AAV particles as described in international patent publication WO 01/92551 (the disclosure of which is incorporated herein by reference in its entirety). Thus, in some embodiments, double stranded (duplex) genomes can be packaged.

As used herein, the term “adeno-associated virus” (AAV), includes but is not limited to, AAV type 1, AAV type 2, AAV type 2.5, AAV type 3 (including types 3A and 3B), AAV type 4, AAV type 5, AAV type 6, AAV type 7, AAV type 8, AAV type 9, AAV type 10, AAV type 11, avian AAV, bovine AAV, canine AAV, equine AAV, ovine AAV, Clade F AAV and any other AAV now known or later discovered. See, e.g., BERNARD N. FIELDS et al., VIROLOGY, volume 2, chapter 69 (4th ed., Lippincott-Raven Publishers). A number of relatively new AAV serotypes and clades have been identified (see Table 1).

The genomic sequences of various serotypes of AAV, as well as the sequences of the native terminal repeats (TRs), Rep proteins, and capsid subunits are known in the art. Exemplary but non-limiting examples of such sequences may be found in the literature or in public databases such as GenBank® Database. See, e.g., GenBank® Database Accession Numbers NC_002077.1, NC_001401.2, NC_001729.1, NC_001863.1, NC_001829.1, NC_006152.1, NC_001862.1, AF513851.1, AF513852.1, the disclosures of which are incorporated by reference herein for teaching parvovirus and AAV nucleic acid and amino acid sequences. See also, e.g., Srivistava et al. (1983) J. Virology 45:555; Chiorini et al. (1998) J. Virology 71:6823; Chiorini et al. (1999) J. Virology 73:1309; Bantel-Schaal et al. (1999) J. Virology 73:939; Xiao et al. (1999) J. Virology 73:3994; Muramatsu et al. (1996) Virology 221:208; Shade et al. (1986) J. Virol. 58:921; Gao et al. (2002) Proc. Nat. Acad. Sci. USA 99:11854; international patent publications WO 00/28061, WO 99/6160 and WO 98/11244; and U.S. Pat. No. 6,156,303; the disclosures of which are incorporated by reference herein for teaching parvovirus and AAV nucleic acid and amino acid sequences.

The capsid structures of autonomous parvoviruses and AAV are described in more detail in BERNARD N. FIELDS et al., Virology, Volume 2, Chapters 69 & 70 (4th ed., Lippincott-Raven Publishers). See also, description of the crystal structure of AAV2 (Xie et al. (2002) Proc. Nat. Acad. Sci. 99:10405-10), AAV4 (Padron et al. (2005) J. Virol. 79: 5047-58), AAV5 (Walters et al. (2004) J. Virol. 78: 3361-71) and CPV (Xie et al. (1996) J Mol. Biol. 6:497-520 and Tsao et al. (1991) Science 251: 1456-64).

A “rAAV vector genome” or “rAAV genome” is an AAV genome (i.e., vDNA) that comprises one or more heterologous nucleic acid sequences. rAAV vectors generally require only a 145 base inverted terminal repeat (ITR) in cis to generate virus. Typically, the rAAV vector genome will only retain the one or more ITR sequence so as to maximize the size of the transgene that can be efficiently packaged by the vector. The structural and non-structural protein coding sequences may be provided in trans (e.g., from a vector, such as a plasmid, or by stably integrating the sequences into a packaging cell). In embodiments of the invention the rAAV vector genome comprises at least one ITR sequence (e.g., AAV ITR sequence), optionally two ITRs (e.g., two AAV ITRs), which typically will be at the 5′ and 3′ ends of the vector genome and flank the heterologous nucleic acid, but need not be contiguous thereto. The ITRs can be the same or different from each other.

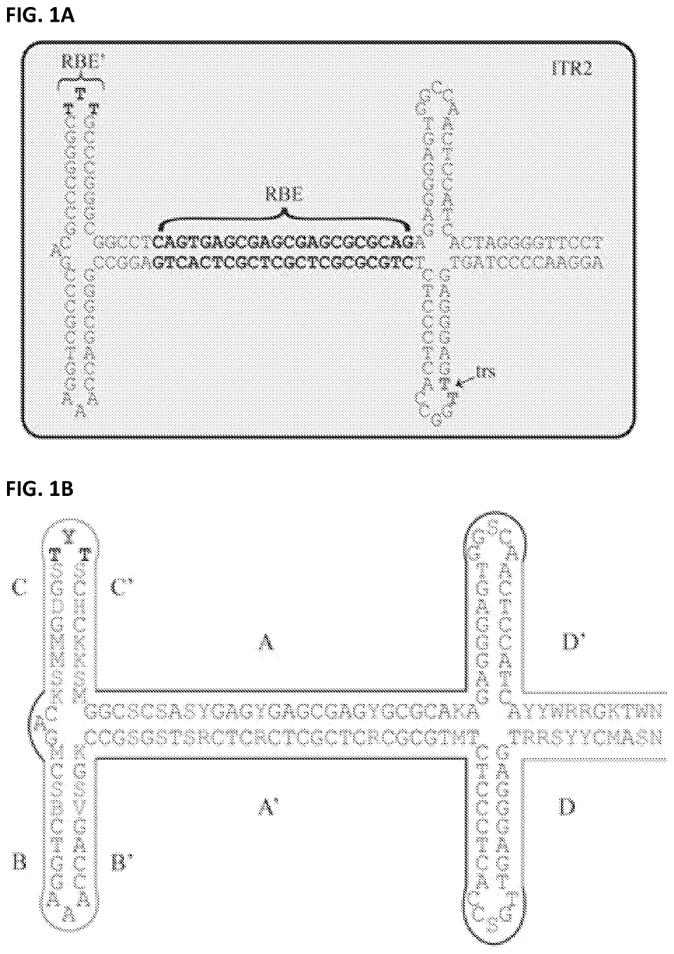

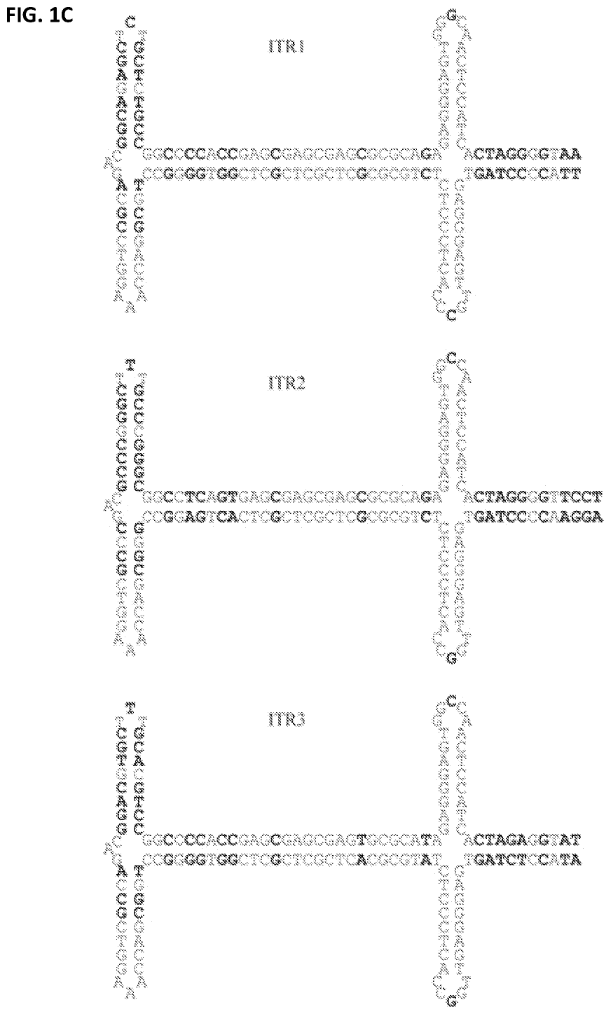

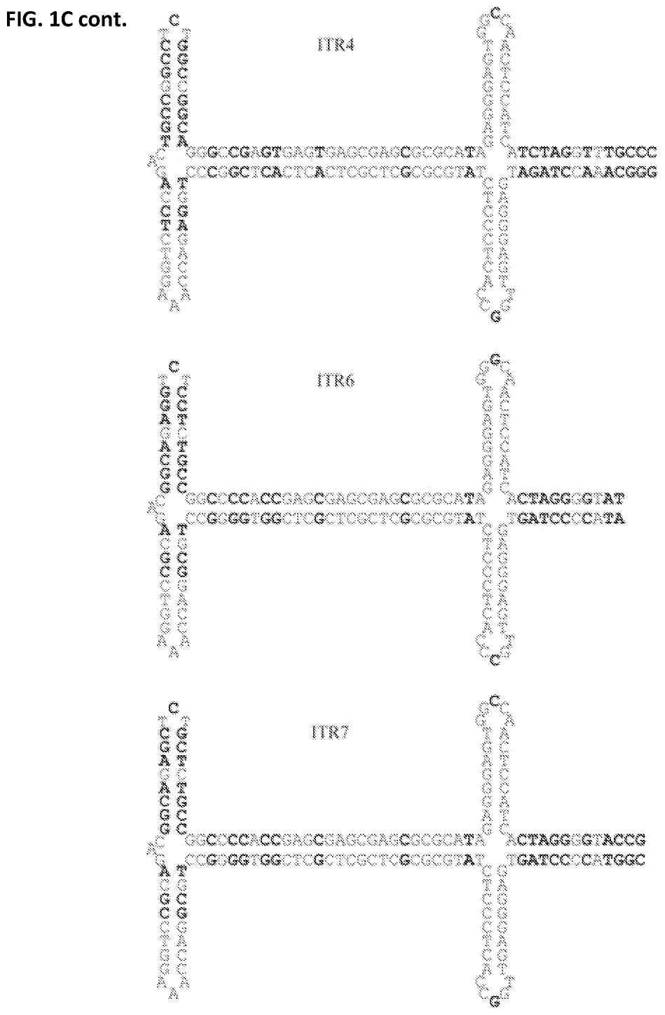

An “AAV inverted terminal repeat” or “AAV ITR” may be from any AAV, including but not limited to serotypes 1, 2, 3a, 3b, 4, 5, 6, 7, 8, 9, 10, 11, or 13, snake AAV, avian AAV, bovine AAV, canine AAV, equine AAV, ovine AAV, goat AAV, shrimp AAV, or any other AAV now known or later discovered (see, e.g., Table 1). An AAV ITR need not have the native terminal repeat sequence (e.g., a native AAV ITR sequence may be altered by insertion, deletion, truncation and/or missense mutations), as long as the terminal repeat mediates the desired functions, e.g., replication, virus packaging, persistence, and/or provirus rescue, and the like, and comprises structural components required for function (e.g., such as depicted in FIGS. 1A-1C.

The virus vectors of the invention can further be “targeted” virus vectors (e.g., having a directed tropism) and/or a “hybrid” AAV (i.e., in which the viral ITRs and viral capsid are from different AAV) as described in international patent publication WO 00/28004 and Chao et al., (2000) Mol. Therapy 2:619.

Further, the viral capsid or genomic elements can contain other modifications, including insertions, deletions and/or substitutions.

The term “template” or “substrate” is used herein to refer to a polynucleotide sequence that may be replicated to produce the AAV viral DNA. For the purpose of vector production, the template will typically be embedded within a larger nucleotide sequence or construct, including but not limited to a plasmid, naked DNA vector, bacterial artificial chromosome (BAC), yeast artificial chromosome (YAC) or a viral vector (e.g., adenovirus, herpesvirus, Epstein-Barr Virus, AAV, baculoviral, retroviral vectors, and the like). Alternatively, the template may be stably incorporated into the chromosome of a packaging cell.

As used herein, AAV “Rep coding sequences” indicate the nucleic acid sequences that encode the AAV non-structural proteins that mediate viral replication and the production of new virus particles. The AAV replication genes and proteins have been described in, e.g., FIELDS et al. VIROLOGY, volume 2, chapters 69 & 70 (4th ed., Lippincott-Raven Publishers).

The “Rep coding sequences” need not encode all of the AAV Rep proteins. For example, with respect to AAV, the Rep coding sequences do not need to encode all four AAV Rep proteins (Rep78, Rep 68, Rep52 and Rep40), in fact, it is believed that AAV5 only expresses the spliced Rep68 and Rep40 proteins. In representative embodiments, the Rep coding sequences encode at least those replication proteins that are necessary for viral genome replication and packaging into new virions. The Rep coding sequences will generally encode at least one large Rep protein (i.e., Rep78/68) and one small Rep protein (i.e., Rep52/40). In particular embodiments, the Rep coding sequences encode the AAV Rep78 protein and the AAV Rep52 and/or Rep40 proteins. In other embodiments, the Rep coding sequences encode the Rep68 and the Rep52 and/or Rep40 proteins. In a still further embodiment, the Rep coding sequences encode the Rep68 and Rep52 proteins, Rep68 and Rep40 proteins, Rep78 and Rep52 proteins, or Rep78 and Rep40 proteins.

As used herein, the term “large Rep protein” refers to Rep68 and/or Rep78. Large Rep proteins of the claimed invention may be either wild-type or synthetic. A wild-type large Rep protein may be from any AAV, including but not limited to serotypes 1, 2, 3a, 3b, 4, 5, 6, 7, 8, 9, 10, 11, or 13, or any other AAV now known or later discovered (see, e.g., Table 1). A synthetic large Rep protein may be altered by insertion, deletion, truncation and/or missense mutations.

Those skilled in the art will further appreciate that it is not necessary that the replication proteins be encoded by the same polynucleotide. For example, for AAV, the p19 promoter may be inactivated and the large Rep protein(s) expressed from one polynucleotide and the small Rep protein(s) expressed from a different polynucleotide. Typically, however, it will be more convenient to express the replication proteins from a single construct. In some systems, the viral promoters (e.g., AAV p19 promoter) may not be recognized by the cell, and it is therefore necessary to express the large and small Rep proteins from separate expression cassettes. In other instances, it may be desirable to express the large Rep and small Rep proteins separately, i.e., under the control of separate transcriptional and/or translational control elements. For example, it may be desirable to control expression of the large Rep proteins, so as to decrease the ratio of large to small Rep proteins. In the case of insect cells, it may be advantageous to down-regulate expression of the large Rep proteins (e.g., Rep78/68) to avoid toxicity to the cells (see, e.g., Urabe et al., (2002) Human Gene Therapy 13:1935).

As used herein, the AAV “cap coding sequences” encode the structural proteins that form a functional AAV capsid (i.e., can package DNA and infect target cells). Typically, the cap coding sequences will encode all of the AAV capsid subunits, but less than all of the capsid subunits may be encoded as long as a functional capsid is produced. Typically, but not necessarily, the cap coding sequences will be present on a single nucleic acid molecule.

The capsid structure of AAV are described in more detail in BERNARD N FIELDS et al., VIROLOGY, volume 2, chapters 69 & 70 (4th ed., Lippincott-Raven Publishers).

As used herein, the term “synthetic AAV ITR” refers to a non-naturally occurring ITR that differs in nucleotide sequence from wild-type ITRs, e.g., the AAV serotype 2 ITR (ITR2) sequence due to one or more deletions, additions, substitutions, or any combination thereof. The difference between the synthetic and wild-type ITR (e.g., ITR2) sequences may be as little as a single nucleotide change, e.g., a change in 1, 2, 3, 4, 5, 6, 7, 8, 9, 10, 15, 20, 25, 30, 35, 40, 45, 50, 60, 60, 70, 80, 90, or 100 or more nucleotides or any range therein. In some embodiments, the difference between the synthetic and wild-type ITR (e.g., ITR2) sequences may be no more than about 100, 90, 80, 70, 60, 50, 45, 40, 35, 30, 25, 20, 15, 10, 9, 8, 7, 6, 5, 4, 3, 2, or 1 nucleotide or any range therein.

The present invention provides polynucleotides comprising at least one AAV ITR that have desirable characteristics and can be designed to manipulate the activities of and cellular responses to vectors comprising the ITR.

Thus, one aspect of the invention relates to polynucleotide comprising at least one synthetic adeno-associated virus (AAV) inverted terminal repeat (ITR), wherein said ITR comprises: (a) an AAV rep binding element (RBE); (b) a B-loop; (c) a C-loop; (d) one or more nicking-stem loops; (e) a D-region; (f) an AAV terminal resolution sequence; and (g) an AAV RBE′ element; wherein (a)-(g) are from any AAV serotype that is not AAV2 or AAV3, and (i) wherein the RBE′ element comprises a non-complementary loop TTT sequence at a position that corresponds to nucleotide positions 73 to 75, wherein the nucleotide numbering is based on the nucleotide sequence of SEQ ID NO:1; (ii) wherein the B-loop comprises a nucleotide sequence that has 80% sequence identity to the nucleotide sequence of the B-loop of ITR2 or ITR3 (i.e., the ITR of AAV2 or AAV3, respectively) at a position that corresponds to nucleotide positions 43-61, wherein the nucleotide numbering is based on the nucleotide sequence of SEQ ID NO:1; (iii) wherein the C-loop comprises a nucleotide sequence that has 80% sequence identity to the nucleotide sequence of the C-loop of ITR2 or ITR3 at a position that corresponds to nucleotide positions 65-83, wherein the nucleotide numbering is based on the nucleotide sequence of SEQ ID NO:1; (iv) wherein the D-region comprises a nucleotide sequence that has 80% sequence identity to the nucleotide sequence of the D-region of ITR2 or ITR3 at a position that corresponds to nucleotide positions 125-145, wherein the nucleotide numbering is based on the nucleotide sequence of SEQ ID NO:1; and/or (v) wherein at least one of the one or more nicking-stem loops comprises a C or G substitution at a position that corresponds to nucleotide position 4 and/or a C or G substitution at a position that corresponds to nucleotide position 122 (e.g., C4G and/or G122C), wherein the nucleotide numbering is based on the nucleotide sequence of SEQ ID NO:1.

SEQ ID NO: 1. AAV2 ITR (ITR2) (NCBI Reference
Sequence NC_001401.2)
TTGGCCACTCCCTCTCTGCGCGCTCGCTCGCTCACTGAGGCCGGGCGAC
CAAAGGTCGCCCGACGCCCGGGCTTTGCCCGGGCGGCCTCAGTGAGCGA
GCGAGCGCGCAGAGAGGGAGTGGCCAACTCCATCACTAGGGGTTCCT

A wildtype (e.g., unmodified) AAV serotype ITR such as depicted in FIGS. 1A-1C comprises multiple structural features including, but not limited to, an A region, a Rep binding element (RBE), a RBE′, one or more nicking-stem loops, a terminal resolution site (trs), a B-loop, a C-loop, and a D-region.

The ITR sequence is predicted to fold back upon itself to form hairpin structures such including regions termed the B-loop and the C-loop (FIGS. 1A-1B). The RBE in the A region of the ITR is a binding site for large Rep proteins (Rep78, Rep68), which can initiate genome replication upon binding the RBE. This initial binding helps to unwind the DNA strands and form a nicking stem that is cleavable by Rep at a dinucleotide TT terminal resolution site (trs). The large Rep proteins also make contact with the RBE′ region at the tip of the C-loop (FIGS. 1A-1B).

The sequence of the RBE, terminal resolution sequence, and RBE′ element of AAV ITRs are well known in the art. The elements in AAV2 ITR are shown in FIG. 1A. Each of the elements as present in the polynucleotide of the present invention can be the exact sequence as exists in a naturally occurring AAV ITR or can differ slightly (e.g., differ by addition, deletion, and/or substitution of no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides) as long as the function of the element is not substantially different from the function of the element as it exists in the naturally occurring AAV ITR. The term “substantially different” is defined herein as a difference in function (e.g., transduction efficiency, Rep binding, nicking) of greater than 50%. The terms RBE, terminal resolution sequence, and RBE′ element as defined herein encompass fragments and portions of the full length elements that provide a function that is not substantially different from the function of the element as it exists in the naturally occurring AAV ITR.

In some embodiments, a synthetic ITR of the present invention, e.g., a synthetic ITR comprising (a) an AAV RBE; (b) a B-loop; (c) a C-loop; (d) one or more nicking-stem loops; (e) a D-region; (f) an AAV terminal resolution sequence; and (g) an AAV RBE′ element; wherein (a)-(g) are from any AAV serotype that is not AAV2 or AAV3, and (i) wherein the RBE′ element comprises a non-complementary loop TTT sequence at a position that corresponds to nucleotide positions 73 to 75, wherein the nucleotide numbering is based on the nucleotide sequence of SEQ ID NO:1; (ii) wherein the B-loop comprises a nucleotide sequence that has 80% sequence identity to the nucleotide sequence of the B-loop of ITR2 or ITR3 (i.e., the ITR of AAV2 or AAV3, respectively) at a position that corresponds to nucleotide positions 43-61, wherein the nucleotide numbering is based on the nucleotide sequence of SEQ ID NO:1; (iii) wherein the C-loop comprises a nucleotide sequence that has 80% sequence identity to the nucleotide sequence of the C-loop of ITR2 or ITR3 at a position that corresponds to nucleotide positions 65-83, wherein the nucleotide numbering is based on the nucleotide sequence of SEQ ID NO:1; (iv) wherein the D-region comprises a nucleotide sequence that has 80% sequence identity to the nucleotide sequence of the D-region of ITR2 or ITR3 at a position that corresponds to nucleotide positions 125-145, wherein the nucleotide numbering is based on the nucleotide sequence of SEQ ID NO:1; and/or (v) wherein at least one of the one or more nicking-stem loops comprises a C or G substitution at a position that corresponds to nucleotide position 4 and/or a C or G substitution at a position that corresponds to nucleotide position 122 (e.g., C4G and/or G122C), wherein the nucleotide numbering is based on the nucleotide sequence of SEQ ID NO:1) may have enhanced transcription function over wildtype (e.g., unmodified) AAV serotype ITR.

Another aspect of the present invention provides a polynucleotide comprising at least one synthetic adeno-associated virus (AAV) inverted terminal repeat (ITR), wherein said ITR comprises: (a) an AAV RBE; (b) a B-loop; (c) a C-loop; (d) one or more nicking-stem loops; (e) a D-region; (f) an AAV terminal resolution sequence; and (g) an AAV RBE′ element; wherein (a)-(g) are from any AAV serotype that is not AAV1 or AAV6, and (i) wherein the RBE′ element comprises a non-complementary loop TCT sequence at a position that corresponds to nucleotide positions 73 to 75, wherein the nucleotide numbering is based on the nucleotide sequence of SEQ ID NO:1; (ii) wherein the B-loop comprises a nucleotide sequence that has 80% sequence identity to the nucleotide sequence of the B-loop of ITR1 or ITR6 (i.e., the ITR of AAV1 or AAV6) at a position that corresponds to nucleotide positions 43-61, wherein the nucleotide numbering is based on the nucleotide sequence of SEQ ID NO:1; (iii) wherein the C-loop comprises a nucleotide sequence that has 80% sequence identity to the nucleotide sequence of the C-loop of ITR1 or ITR6 at a position that corresponds to nucleotide positions 65-83, wherein the nucleotide numbering is based on the nucleotide sequence of SEQ ID NO:1; (iv) wherein the D-region comprises a nucleotide sequence that has 80% sequence identity to the nucleotide sequence of the D-region of ITR1 or ITR6 at a position that corresponds to nucleotide positions 125-145, wherein the nucleotide numbering is based on the nucleotide sequence of SEQ ID NO:1; and/or (v) wherein at least one of the one or more nicking-stem loops comprises a C or G substitution at a position that corresponds to nucleotide position 4 and/or a C or G substitution at a position that corresponds to nucleotide position 122 (e.g., G4C and/or C122G), wherein the nucleotide numbering is based on the nucleotide sequence of SEQ ID NO:1.

In some embodiments, said synthetic ITR (e.g., a synthetic ITR comprising (a) an AAV RBE; (b) a B-loop; (c) a C-loop; (d) one or more nicking-stem loops; (e) a D-region; (f) an AAV terminal resolution sequence; and (g) an AAV RBE′ element; wherein (a)-(g) are from any AAV serotype that is not AAV1 or AAV6, and (i) wherein the RBE′ element comprises a non-complementary loop TCT sequence at a position that corresponds to nucleotide positions 73 to 75, wherein the nucleotide numbering is based on the nucleotide sequence of SEQ ID NO:1; (ii) wherein the B-loop comprises a nucleotide sequence that has 80% sequence identity to the nucleotide sequence of the B-loop of ITR1 or ITR6 (i.e., the ITR of AAV1 or AAV6) at a position that corresponds to nucleotide positions 43-61, wherein the nucleotide numbering is based on the nucleotide sequence of SEQ ID NO:1; (iii) wherein the C-loop comprises a nucleotide sequence that has 80% sequence identity to the nucleotide sequence of the C-loop of ITR1 or ITR6 at a position that corresponds to nucleotide positions 65-83, wherein the nucleotide numbering is based on the nucleotide sequence of SEQ ID NO:1; (iv) wherein the D-region comprises a nucleotide sequence that has 80% sequence identity to the nucleotide sequence of the D-region of ITR1 or ITR6 at a position that corresponds to nucleotide positions 125-145, wherein the nucleotide numbering is based on the nucleotide sequence of SEQ ID NO:1; and/or (v) wherein at least one of the one or more nicking-stem loops comprises a C or G substitution at a position that corresponds to nucleotide position 4 and/or a C or G substitution at a position that corresponds to nucleotide position 122 (e.g., G4C and/or C122G), wherein the nucleotide numbering is based on the nucleotide sequence of SEQ ID NO:1) may have reduced transcription function over wildtype (e.g., unmodified) AAV serotype ITR.

The structural features (a)-(g) of a synthetic ITR of the present invention may be from any AAV serotype, such as, but not limited to, any AAV serotype of Table 1. In some embodiments, (a)-(g) of a synthetic ITR of the present invention are from the same AAV. In some embodiments, (a)-(g) of a synthetic ITR of the present invention are from different AAV. Production of the polynucleotides and/or synthetic ITRs of this invention can be carried out by introducing some (e.g., 1, 2, 3, 4, 5, 6, 7, 8, 9, 10, 11, 12, 13, 14, 15, etc.) or all of the modifications identified from another AAV serotype, as identified, for example, in FIGS. 1A-1C. Not every nucleotide identified as part of a particular region of an AAV ITR is required to be substituted to produce the synthetic ITR of this invention. The number of substitutions necessary to produce the desired functional and/or structural ITR properties can be readily determined by one of ordinary skill in the art according to the teachings herein and according to protocols well known in the art. The nucleotide numbering provided in the nucleotide sequences set forth here is based on the unmodified (e.g., wild type) nucleotide sequence of the ITR of AAV2 (SEQ ID NO:1) as provided herein. However it would be readily understood by one of ordinary skill in the art that the equivalent nucleotide positions in other AAV serotype ITR sequences or other viral ITR sequences can be readily identified and employed in the production of the polynucleotides and/or synthetic ITRs of this invention, and can be identified via alignments such as disclosed herein in the SEQUENCES section and such as disclosed in Hewitt et a., 2009 J. Virol. 83(8):3919-3929, incorporated herein by reference.

In some embodiments, a synthetic ITR of the present invention may further comprise additional non-AAV cis elements, e.g., elements that initiate transcription, mediate enhancer function, allow replication and symmetric distribution upon mitosis, or alter the persistence and processing of transduced genomes. Such elements are well known in the art and include, without limitation, promoters, enhancers, chromatin attachment sequences, telomeric sequences, cis-acting microRNAs, and combinations thereof. In some embodiments, a synthetic ITR of the present invention may explicitly not comprise any additional non-AAV cis elements. For example, in some embodiments, a synthetic ITR of the present invention may not comprise a promoter.

In some embodiments, a polynucleotide of the present invention may further comprise one or more insulator sequence. As used herein, the term “insulator sequence” refers to a sequence (e.g., a sequence outside of the ITR) which may inhibit and/or otherwise attenuate ITR transcriptional activity. Examples of insulator sequences are known in the art.

In some embodiments, a polynucleotide of the present invention may further comprise a heterologous nucleotide sequence (e.g., a coding sequence, e.g., encoding a protein or a functional RNA).

Wildtype (e.g., unmodified) AAV ITR sequences are known to comprise CpG motifs. In some embodiments, one or more CpG motifs in a synthetic ITR of the present invention may be deleted and/or substituted, relative to the sequence of a naturally occurring AAV ITR such as ITR2. The AAV ITR2 contains 16 CpG motifs. TLR-9 directly binds to CpG sequence motifs and results in the activation of cellular innate immunity. It is also well known that methylation of CpG motifs results in transcriptional silencing. Removal of CpG motifs in the ITR may result in decreased TLR-9 recognition and/or decreased methylation and therefore decreased transgene silencing.

In addition, recent studies have shown that double-stranded RNA (dsRNA) may be sensed by dsRNA sensors MDA5 or MAVS and trigger an immune response, as described in Shao et al. 2018 JCI Insight 3(12):e120474, and Faust et al. 2013 JCI 123(7):2994-3001, incorporated herein by reference in their entireties. While not wishing to be bound to theory, removal/reduction of CpG motifs in the ITR may result in decreased dsRNA immune response during the use of gene therapy comprising a polynucleotide and/or synthetic ITR of the present invention.

In some embodiments at least 1 CpG motif is deleted and/or substituted, e.g., at least 4 or more or 8 or more CpG motifs, e.g., at least 1, 2, 3, 4, 5, 6, 7, 8, 9, 10, 11, 12, 13, 14, 15, or 16 CpG motifs. The phrase “deleted and/or substituted” as used herein means that one or both nucleotides in the CpG motif is deleted, substituted with a different nucleotide, or any combination of deletions and substitutions.

The invention also provides a vector comprising the polynucleotide comprising the synthetic ITR of the invention. The viral vector can be a parvovirus vector, e.g., an AAV vector. The invention further provides a recombinant parvovirus particle (e.g., a recombinant AAV particle) comprising the synthetic ITR of the invention. In some embodiments, the invention provides a chimeric AAV particle comprising an ITR from any AAV serotype or the synthetic ITR of the present invention, wherein additional AAV cis elements (e.g., Rep and/or Cap) are from a different AAV serotype than the ITR. Viral vectors and viral particles are discussed further below.

The present invention also provides a composition comprising a virus vector of this invention in a pharmaceutically acceptable carrier.

### Methods of Use

The invention further provides methods of use for the polynucleotides, nucleic acids, synthetic ITRs, viruses, vectors, particles, and/or compositions of this invention.

In one aspect, the present invention provides a method of transcribing a heterologous nucleotide sequence in a cell, comprising introducing into the cell a polynucleotide of the present invention.

In some embodiments, the polynucleotide of the present invention introduced into the cell may further comprise additional non-AAV cis elements, e.g., elements that initiate transcription, mediate enhancer function, allow replication and symmetric distribution upon mitosis, or alter the persistence and processing of transduced genomes, such as, but not limited to, promoters, enhancers, chromatin attachment sequences, telomeric sequences, cis-acting microRNAs, and combinations thereof. In some embodiments, the polynucleotide of the present invention introduced into the cell may explicitly not comprise any additional non-AAV cis elements. In some embodiments, the polynucleotide introduced into the cell does not comprise additional non-AAV cis promoters.

Further provided is a method of delivering a nucleic acid to a cell, comprising introducing into a cell a recombinant AAV particle of the present invention.

Another aspect of the present invention provides a method of producing a recombinant AAV particle, comprising providing to a cell permissive for AAV replication: (a) a recombinant AAV template comprising (i) a heterologous nucleic acid, and (ii) a synthetic ITR of the present invention; and (b) a polynucleotide comprising Rep coding sequences and Cap coding sequences; under conditions sufficient for the replication and packaging of the recombinant AAV template; whereby recombinant AAV particles are produced in the cell.

Another aspect of the present invention provides a method of producing a recombinant AAV particle, comprising providing to a cell permissive for AAV replication: (a) a recombinant AAV template comprising (i) a heterologous nucleic acid, and (ii) a wildtype ITR from any AAV serotype or a synthetic ITR of the present invention; and (b) a polynucleotide comprising Rep coding sequences and Cap coding sequences, wherein the Rep and Cap coding sequences are from a different AAV serotype; under conditions sufficient for the replication and packaging of the recombinant AAV template; whereby recombinant AAV particles are produced in the cell.

In some embodiments, the Rep coding sequences and Cap coding sequences cannot be packaged into the recombinant AAV particle.

In some embodiments, the Rep coding sequences and/or Cap coding sequences may be provided by a plasmid.

In some embodiments, the Rep coding sequences and/or Cap coding sequences may be provided by a viral vector. The viral vector may be any viral vector known in the art, including, but not limited to, an adenovirus vector, herpesvirus vector, Epstein-Barr virus vector, and baculovirus vector.

In some embodiments, the Rep coding sequences may be stably integrated into the genome of the cell.

In some embodiments, the Cap coding sequences may be stably integrated into the genome of the cell.

In some embodiments, the recombinant AAV template may be provided by a plasmid and/or a viral vector or may be stably integrated into the genome of the cell as a provirus.

Further provided is a method of administering a nucleic acid to a mammalian subject comprising administering to the mammalian subject a cell that has been contacted with a recombinant AAV particle of the present invention under conditions sufficient for the AAV particle vector genome to enter the cell. Non-limiting examples of a target cell include a neural cell, lung cell, retinal cell, epithelial cell, smooth muscle cell, skeletal muscle cell, cardiac muscle cell, pancreatic cell, hepatic cell, kidney cell, myocardial cell, bone cell, spleen cell, keratinocyte, fibroblast, endothelial cell, prostate cell, germ cell, progenitor cell, stem cell, cancer cell, and tumor cell.

Additionally provided is a method of administering a nucleic acid to a mammalian subject comprising administering to the mammalian subject a recombinant AAV particle of the present invention. In some embodiments, the recombinant AAV particle may be administered to the mammalian subject in a dose range from about 1×10⁶ vg/kg to about 1×10¹⁵ vg/kg, e.g., about 1×10⁶, 1×10⁷, 1×10⁸, 1×10⁹, 1×10¹⁰, 1×10¹¹, 1×10¹², 1×10¹³, 1×10¹⁴, 1×10¹⁵ vg/kg or any value or range therein. For example, in some embodiments, the recombinant AAV particle may be administered to the mammalian subject in a dose range from about 1×10⁶ vg/kg to about 5×10¹² vg/kg, about 5×10¹⁰ vg/kg to about 1×10¹⁵ vg/kg, or about 1×10⁶ vg/kg, about 5×10⁸ vg/kg, about 5×10¹⁰ vg/kg, about 5×10¹² vg/kg, or about 1×10¹⁴ vg/kg.

In some embodiments, the mammalian subject may be a human subject.

In some embodiments, the AAV particle may be administered by a route selected from the group consisting of oral, rectal, transmucosal, transdermal, inhalation, intravenous, subcutaneous, intradermal, intracranial, intramuscular, intraendothelial, intravitreal, subretinal, intraarticular administration, and any combination thereof.

In some embodiments, the AAV particle may be administered to the subject at a site selected from the group consisting of a tumor, the brain, a skeletal muscle, a smooth muscle, the heart, the diaphragm, the airway epithelium, the liver, the kidney, the spleen, the pancreas, the skin, the eye, and any combination thereof.

In some embodiments, the mammalian subject may have a reduced immune response against the AAV particle as compared to an AAV particle which does not comprise a polynucleotide of the present invention.

Further provided herein is a method of enhancing promoter function of an adeno-associated virus (AAV) inverted terminal repeat (ITR) relative to a wildtype (e.g., unmodified) ITR, wherein said ITR comprises: (a) an AAV rep binding element (RBE); (b) a B-loop; (c) a C-loop; (d) one or more nicking-stem loops; (e) a D-region; (f) an AAV terminal resolution sequence; and (g) an AAV RBE′ element; wherein (a)-(g) are from any AAV serotype that is not AAV2 or AAV3, comprising substituting one or more of the following: (i) a non-complementary loop TTT sequence at a position that corresponds to nucleotide positions 73 to 75, wherein the nucleotide numbering is based on the nucleotide sequence of SEQ ID NO:1; (ii) a nucleotide sequence that has 80% sequence identity to the nucleotide sequence of the B-loop of ITR2 or ITR3 (i.e., the ITR of AAV2 or AAV3, respectively) at a position that corresponds to nucleotide positions 43-61, wherein the nucleotide numbering is based on the nucleotide sequence of SEQ ID NO:1; (iii) a nucleotide sequence that has 80% sequence identity to the nucleotide sequence of the C-loop of ITR2 or ITR3 at nucleotide positions 65-83, wherein the nucleotide numbering is based on the nucleotide sequence of SEQ ID NO:1; (iv) a nucleotide sequence that has 80% sequence identity to the nucleotide sequence of the D-region of ITR2 or ITR3 at nucleotide positions 125-145, wherein the nucleotide numbering is based on the nucleotide sequence of SEQ ID NO:1; and/or (v) a G or C substitution at nucleotide position 4 and/or a G or C substitution at nucleotide position 122 (e.g., C4G and/or G122C), wherein the nucleotide numbering is based on the nucleotide sequence of SEQ ID NO:1.

Another aspect of the present invention provides a method of reducing promoter function of an adeno-associated virus (AAV) inverted terminal repeat (ITR) relative to a wildtype (e.g., unmodified) ITR, wherein said ITR comprises: (a) an AAV rep binding element (RBE); (b) a B-loop; (c) a C-loop; (d) one or more nicking-stem loops; (e) a D-region; (f) an AAV terminal resolution sequence; and (g) an AAV RBE′ element; wherein (a)-(g) are from any AAV serotype that is not AAV1 or AAV6, comprising substituting one or more of the following: (i) a non-complementary loop TCT sequence at a position that corresponds to nucleotide positions 73 to 75, wherein the nucleotide numbering is based on the nucleotide sequence of SEQ ID NO:1; (ii) a nucleotide sequence that has 80% sequence identity to the nucleotide sequence of the B-loop of ITR1 or ITR6 (i.e., the ITR of AAV1 or AAV6) at nucleotide positions 43-61, wherein the nucleotide numbering is based on the nucleotide sequence of SEQ ID NO:1; (iii) a nucleotide sequence that has 80% sequence identity to the nucleotide sequence of the C-loop of ITR1 or ITR6 at nucleotide positions 65-83, wherein the nucleotide numbering is based on the nucleotide sequence of SEQ ID NO:1; (iv) a nucleotide sequence that has 80% sequence identity to the nucleotide sequence of the D-region of ITR1 or ITR6 at nucleotide positions 125-145, wherein the nucleotide numbering is based on the nucleotide sequence of SEQ ID NO:1; and/or (v) a C substitution at nucleotide position 4 and/or a G substitution at nucleotide position 122 (e.g., G4C and/or C122G), wherein the nucleotide numbering is based on the nucleotide sequence of SEQ ID NO:1.

In some embodiments, the (unmodified) ITR prior to modification had high transcriptional activity.

In some embodiments, the (unmodified) ITR prior to modification had low transcriptional activity.

In some embodiments, the methods of the present invention further comprise modifying CpG island and/or C-G dinucleotide frequency within the ITR (e.g., inserting one or more [e.g., 1, 2, 3, 4, 5, 6, 7, 8, 9, 10 etc. or more) CpG islands and/or C-G dinucleotide sequences and/or deleting one or more [e.g., 1, 2, 3, 4, 5, 6, 7, 8, 9, 10 etc. or more) CpG islands and/or C-G dinucleotide sequences). While not wishing to be bound to theory, modifying the CpG and/or C-G dinucleotide frequency may modify the transcriptional activity of the synthetic ITR. In some embodiments, the synthetic ITR of the present invention has at least one or more GpC and/or C-G dinucleotide sequences and retains functional activity of a wildtype (unmodified) ITR.

In some embodiments, the methods of the present invention further comprise wherein ITR bidirectional activity is attenuated and immune response is mitigated. While not wishing to be bound to theory, it is believed that ITR bidirectional activity may enhance dsRNA recognition and immune response triggering, as described in Shao et al. 2018 JCI Insight 3(12):e120474, incorporated herein by reference.

### Methods of Producing Virus Vectors

The present invention further provides methods of producing virus vectors.

In one embodiment, the present invention provides a method of producing a recombinant AAV particle, comprising providing to a cell permissive for AAV replication: (a) a recombinant AAV template comprising (i) a heterologous nucleotide sequence, and (ii) the synthetic ITR of the invention; (b) a polynucleotide comprising Rep coding sequences and Cap coding sequences; under conditions sufficient for the replication and packaging of the recombinant AAV template; whereby recombinant AAV particles are produced in the cell.

Conditions sufficient for the replication and packaging of the recombinant AAV template can be, e.g., the presence of AAV sequences sufficient for replication of the AAV template and encapsidation into AAV capsids (e.g., AAV rep sequences and AAV cap sequences) and helper sequences from adenovirus and/or herpesvirus. In particular embodiments, the AAV template comprises two AAV ITR sequences, which are located 5′ and 3′ to the heterologous nucleic acid sequence, although they need not be directly contiguous thereto.

In some embodiments, the recombinant AAV template comprises an ITR that is not resolved by Rep to make duplexed AAV vectors as described in international patent publication WO 01/92551.

The nucleic acid template and AAV rep and cap sequences are provided under conditions such that virus vector comprising the nucleic acid template packaged within the AAV capsid is produced in the cell. The method can further comprise the step of collecting the virus vector from the cell. The virus vector can be collected from the medium and/or by lysing the cells.

The cell can be a cell that is permissive for AAV viral replication. Any suitable cell known in the art may be employed. In particular embodiments, the cell is a mammalian cell. As another option, the cell can be a trans-complementing packaging cell line that provides functions deleted from a replication-defective helper virus, e.g., 293 cells or other Ela trans-complementing cells.

The AAV replication and capsid sequences may be provided by any method known in the art. Current protocols typically express the AAV rep/cap genes on a single plasmid. The AAV replication and packaging sequences need not be provided together, although it may be convenient to do so. The AAV rep and/or cap sequences may be provided by any viral or non-viral vector. For example, the rep/cap sequences may be provided by a hybrid adenovirus or herpesvirus vector (e.g., inserted into the Ela or E3 regions of a deleted adenovirus vector). EBV vectors may also be employed to express the AAV cap and rep genes. One advantage of this method is that EBV vectors are episomal, yet will maintain a high copy number throughout successive cell divisions (i.e., are stably integrated into the cell as extra-chromosomal elements, designated as an “EBV based nuclear episome.”

As a further alternative, the rep/cap sequences may be stably incorporated into a cell.

Typically the AAV rep/cap sequences will not be flanked by the TRs, to prevent rescue and/or packaging of these sequences.

The nucleic acid template can be provided to the cell using any method known in the art. For example, the template can be supplied by a non-viral (e.g., plasmid) or viral vector. In particular embodiments, the nucleic acid template is supplied by a herpesvirus or adenovirus vector (e.g., inserted into the E1a or E3 regions of a deleted adenovirus). As another example, a baculovirus vector carrying a reporter gene flanked by the AAV TRs can be used. EBV vectors may also be employed to deliver the template, as described above with respect to the rep/cap genes.

In another representative embodiment, the nucleic acid template is provided by a replicating rAAV virus. In still other embodiments, an AAV provirus comprising the nucleic acid template is stably integrated into the chromosome of the cell.

To enhance virus titers, helper virus functions (e.g., adenovirus or herpesvirus) that promote a productive AAV infection can be provided to the cell. Helper virus sequences necessary for AAV replication are known in the art. Typically, these sequences will be provided by a helper adenovirus or herpesvirus vector. Alternatively, the adenovirus or herpesvirus sequences can be provided by another non-viral or viral vector, e.g., as a non-infectious adenovirus miniplasmid that carries all of the helper genes that promote efficient AAV production.

Further, the helper virus functions may be provided by a packaging cell with the helper sequences embedded in the chromosome or maintained as a stable extrachromosomal element. Generally, the helper virus sequences cannot be packaged into AAV virions, e.g., are not flanked by TRs.

Those skilled in the art will appreciate that it may be advantageous to provide the AAV replication and capsid sequences and the helper virus sequences (e.g., adenovirus sequences) on a single helper construct. This helper construct may be a non-viral or viral construct. As one nonlimiting illustration, the helper construct can be a hybrid adenovirus or hybrid herpesvirus comprising the AAV rep/cap genes.

In one particular embodiment, the AAV rep/cap sequences and the adenovirus helper sequences are supplied by a single adenovirus helper vector. This vector can further comprise the nucleic acid template. The AAV rep/cap sequences and/or the rAAV template can be inserted into a deleted region (e.g., the Ela or E3 regions) of the adenovirus.

In a further embodiment, the AAV rep/cap sequences and the adenovirus helper sequences are supplied by a single adenovirus helper vector. According to this embodiment, the rAAV template can be provided as a plasmid template.

In another illustrative embodiment, the AAV rep/cap sequences and adenovirus helper sequences are provided by a single adenovirus helper vector, and the rAAV template is integrated into the cell as a provirus. Alternatively, the rAAV template is provided by an EBV vector that is maintained within the cell as an extrachromosomal element (e.g., as an EBV based nuclear episome).

In a further exemplary embodiment, the AAV rep/cap sequences and adenovirus helper sequences are provided by a single adenovirus helper. The rAAV template can be provided as a separate replicating viral vector. For example, the rAAV template can be provided by a rAAV particle or a second recombinant adenovirus particle.

According to the foregoing methods, the hybrid adenovirus vector typically comprises the adenovirus 5′ and 3′ cis sequences sufficient for adenovirus replication and packaging (i.e., the adenovirus terminal repeats and PAC sequence). The AAV rep/cap sequences and, if present, the rAAV template are embedded in the adenovirus backbone and are flanked by the 5′ and 3′ cis sequences, so that these sequences may be packaged into adenovirus capsids. As described above, the adenovirus helper sequences and the AAV rep/cap sequences are generally not flanked by TRs so that these sequences are not packaged into the AAV virions.

Zhang et al. ((2001) Gene Ther. 18:704-12) describes a chimeric helper comprising both adenovirus and the AAV rep and cap genes.

Herpesvirus may also be used as a helper virus in AAV packaging methods. Hybrid herpesviruses encoding the AAV Rep protein(s) may advantageously facilitate scalable AAV vector production schemes. A hybrid herpes simplex virus type I (HSV-1) vector expressing the AAV-2 rep and cap genes has been described, e.g., PCT Publication No. WO 00/17377, incorporated by reference herein.

As a further alternative, the virus vectors of the invention can be produced in insect cells using baculovirus vectors to deliver the rep/cap genes and rAAV template.

AAV vector stocks free of contaminating helper virus may be obtained by any method known in the art. For example, AAV and helper virus may be readily differentiated based on size. AAV may also be separated away from helper virus based on affinity for a heparin substrate. Deleted replication-defective helper viruses can be used so that any contaminating helper virus is not replication competent. As a further alternative, an adenovirus helper lacking late gene expression may be employed, as only adenovirus early gene expression is required to mediate packaging of AAV virus. Adenovirus mutants defective for late gene expression are known in the art (e.g., ts100K and ts149 adenovirus mutants).

### Recombinant Virus Vectors

The virus vectors of the present invention are useful for the delivery of nucleic acids to cells in vitro, ex vivo, and in vivo. In particular, the virus vectors can be advantageously employed to deliver or transfer nucleic acids to animal cells, including e.g., mammalian cells.

Any heterologous nucleic acid sequence(s) of interest may be delivered in the virus vectors of the present invention. Nucleic acids of interest include nucleic acids encoding polypeptides, including therapeutic (e.g., for medical or veterinary uses) and/or immunogenic (e.g., for vaccines) polypeptides.

Therapeutic polypeptides include, but are not limited to, cystic fibrosis transmembrane regulator protein (CFTR), dystrophin (including mini- and micro-dystrophins, see, e.g., Vincent et al. (1993) Nature Genetics 5:130; U.S. Patent Publication No. 2003017131; PCT Publication No. WO/2008/088895, Wang et al. Proc. Natl. Acad. Sci. USA 97:13714-13719 (2000); and Gregorevic et al. Mol. Ther. 16:657-64 (2008)), myostatin propeptide, follistatin, activin type II soluble receptor, IGF-1, anti-inflammatory polypeptides such as the I kappa B dominant mutant, sarcospan, utrophin (Tinsley et al. (1996) Nature 384:349), mini-utrophin, clotting factors (e.g., Factor VIII, Factor IX, Factor X, etc.), erythropoietin, angiostatin, endostatin, catalase, tyrosine hydroxylase, superoxide dismutase, leptin, the LDL receptor, lipoprotein lipase, ornithine transcarbamylase, β-globin, α-globin, spectrin, α₁-antitrypsin, adenosine deaminase, hypoxanthine guanine phosphoribosyl transferase, β-glucocerebrosidase, sphingomyelinase, lysosomal hexosaminidase A, branched-chain keto acid dehydrogenase, RP65 protein, cytokines (e.g., α-interferon, β-interferon, interferon-γ, interleukin-2, interleukin-4, granulocyte-macrophage colony stimulating factor, lymphotoxin, and the like), peptide growth factors, neurotrophic factors and hormones (e.g., somatotropin, insulin, insulin-like growth factors 1 and 2, platelet derived growth factor, epidermal growth factor, fibroblast growth factor, nerve growth factor, neurotrophic factor-3 and -4, brain-derived neurotrophic factor, bone morphogenic proteins [including RANKL and VEGF], glial derived growth factor, transforming growth factor-α and -β, and the like), lysosomal acid α-glucosidase, α-galactosidase A, receptors (e.g., the tumor necrosis growth factora soluble receptor), S100A1, parvalbumin, adenylyl cyclase type 6, a molecule that modulates calcium handling (e.g., SERCA2A, Inhibitor 1 of PP1 and fragments thereof [e.g., PCT Publication Nos. WO 2006/029319 and WO 2007/100465]), a molecule that effects G-protein coupled receptor kinase type 2 knockdown such as a truncated constitutively active bARKct, anti-inflammatory factors such as IRAP, anti-myostatin proteins, aspartoacylase, monoclonal antibodies (including single chain monoclonal antibodies; an exemplary Mab being the Herceptin® Mab), neuropeptides and fragments thereof (e.g., galanin, Neuropeptide Y (see U.S. Pat. No. 7,071,172), angiogenesis inhibitors such as Vasohibins and other VEGF inhibitors (e.g., Vasohibin 2 [see PCT Publication WO JP2006/073052]). Other illustrative heterologous nucleic acid sequences encode suicide gene products (e.g., thymidine kinase, cytosine deaminase, diphtheria toxin, and tumor necrosis factor), proteins conferring resistance to a drug used in cancer therapy, tumor suppressor gene products (e.g., p53, Rb, Wt-1), TRAIL, FAS-ligand, and any other polypeptide that has a therapeutic effect in a subject in need thereof. AAV vectors can also be used to deliver monoclonal antibodies and antibody fragments, for example, an antibody or antibody fragment directed against myostatin (see, e.g., Fang et al. Nature Biotechnology 23:584-590 (2005)).

Heterologous nucleic acid sequences encoding polypeptides include those encoding reporter polypeptides (e.g., an enzyme). Reporter polypeptides are known in the art and include, but are not limited to, green fluorescent protein (GFP), β-galactosidase, alkaline phosphatase, luciferase, and chloramphenicol acetyltransferase gene.

Optionally, the heterologous nucleic acid encodes a secreted polypeptide (e.g., a polypeptide that is a secreted polypeptide in its native state or that has been engineered to be secreted, for example, by operable association with a secretory signal sequence as is known in the art).

Alternatively, in particular embodiments of this invention, the heterologous nucleic acid may encode an antisense nucleic acid, a ribozyme (e.g., as described in U.S. Pat. No. 5,877,022), RNAs that effect spliceosome-mediated trans-splicing (see, Puttaraju et al. (1999) Nature Biotech. 17:246; U.S. Pat. Nos. 6,013,487; 6,083,702), interfering RNAs (RNAi) including siRNA, shRNA or miRNA that mediate gene silencing (see, Sharp et al. (2000) Science 287:2431), and other non-translated RNAs, such as “guide” RNAs (Gorman et al. (1998) Proc. Nat. Acad. Sci. USA 95:4929; U.S. Pat. No. 5,869,248 to Yuan et al.), and the like. Exemplary untranslated RNAs include RNAi against a multiple drug resistance (MDR) gene product (e.g., to treat and/or prevent tumors and/or for administration to the heart to prevent damage by chemotherapy), RNAi against myostatin (e.g., for Duchenne muscular dystrophy), RNAi against VEGF (e.g., to treat and/or prevent tumors), RNAi against phospholamban (e.g., to treat cardiovascular disease, see e.g., Andino et al. J. Gene Med. 10:132-142 (2008) and Li et al. Acta Pharmacol Sin. 26:51-55 (2005)); phospholamban inhibitory or dominant-negative molecules such as phospholamban S16E (e.g., to treat cardiovascular disease, see e.g., Hoshijima et al. Nat. Med. 8:864-871 (2002)), RNAi to adenosine kinase (e.g., for epilepsy), and RNAi directed against pathogenic organisms and viruses (e.g., hepatitis B and/or C virus, human immunodeficiency virus, CMV, herpes simplex virus, human papilloma virus, etc.).

Further, a nucleic acid sequence that directs alternative splicing can be delivered. To illustrate, an antisense sequence (or other inhibitory sequence) complementary to the 5′ and/or 3′ splice site of dystrophin exon 51 can be delivered in conjunction with a U1 or U7 small nuclear (sn) RNA promoter to induce skipping of this exon. For example, a DNA sequence comprising a U1 or U7 snRNA promoter located 5′ to the antisense/inhibitory sequence(s) can be packaged and delivered in a modified capsid of the invention.

The virus vector may also comprise a heterologous nucleic acid that shares homology with and recombines with a locus on a host chromosome. This approach can be utilized, for example, to correct a genetic defect in the host cell.

The present invention also provides virus vectors that express an immunogenic polypeptide, e.g., for vaccination. The nucleic acid may encode any immunogen of interest known in the art including, but not limited to, immunogens from human immunodeficiency virus (HIV), simian immunodeficiency virus (SIV), influenza virus, HIV or SIV gag proteins, tumor antigens, cancer antigens, bacterial antigens, viral antigens, and the like.

The use of parvoviruses as vaccine vectors is known in the art (see, e.g., Miyamura et al., (1994) Proc. Nat. Acad. Sci USA 91:8507; U.S. Pat. No. 5,916,563 to Young et al., U.S. Pat. No. 5,905,040 to Mazzara et al., U.S. Pat. Nos. 5,882,652, 5,863,541 to Samulski et al.). The antigen may be presented in the parvovirus capsid. Alternatively, the antigen may be expressed from a heterologous nucleic acid introduced into a recombinant vector genome. Any immunogen of interest as described herein and/or as is known in the art can be provided by the virus vector of the present invention.

An immunogenic polypeptide can be any polypeptide suitable for eliciting an immune response and/or protecting the subject against an infection and/or disease, including, but not limited to, microbial, bacterial, protozoal, parasitic, fungal and/or viral infections and diseases. For example, the immunogenic polypeptide can be an orthomyxovirus immunogen (e.g., an influenza virus immunogen, such as the influenza virus hemagglutinin (HA) surface protein or the influenza virus nucleoprotein, or an equine influenza virus immunogen) or a lentivirus immunogen (e.g., an equine infectious anemia virus immunogen, a Simian Immunodeficiency Virus (SIV) immunogen, or a Human Immunodeficiency Virus (HIV) immunogen, such as the HIV or SIV envelope GP160 protein, the HIV or SIV matrix/capsid proteins, and the HIV or SIV gag, pol and env gene products). The immunogenic polypeptide can also be an arenavirus immunogen (e.g., Lassa fever virus immunogen, such as the Lassa fever virus nucleocapsid protein and/or the Lassa fever envelope glycoprotein), a poxvirus immunogen (e.g., a vaccinia virus immunogen, such as the vaccinia L1 or L8 gene product), a flavivirus immunogen (e.g., a yellow fever virus immunogen or a Japanese encephalitis virus immunogen), a filovirus immunogen (e.g., an Ebola virus immunogen, or a Marburg virus immunogen, such as NP and GP gene products), a bunyavirus immunogen (e.g., RVFV, CCHF, and/or SFS virus immunogens), or a coronavirus immunogen (e.g., an infectious human coronavirus immunogen, such as the human coronavirus envelope glycoprotein, or a porcine transmissible gastroenteritis virus immunogen, or an avian infectious bronchitis virus immunogen). The immunogenic polypeptide can further be a polio immunogen, a herpesvirus immunogen (e.g., CMV, EBV, HSV immunogens) a mumps virus immunogen, a measles virus immunogen, a rubella virus immunogen, a diphtheria toxin or other diphtheria immunogen, a pertussis antigen, a hepatitis (e.g., hepatitis A, hepatitis B, hepatitis C, etc.) immunogen, and/or any other vaccine immunogen now known in the art or later identified as an immunogen.

Alternatively, the immunogenic polypeptide can be any tumor or cancer cell antigen. Optionally, the tumor or cancer antigen is expressed on the surface of the cancer cell. Exemplary cancer and tumor cell antigens are described in S. A. Rosenberg (Immunity 10:281 (1991)). Other illustrative cancer and tumor antigens include, but are not limited to: BRCA1 gene product, BRCA2 gene product, gp100, tyrosinase, GAGE-1/2, BAGE, RAGE, LAGE, NY-ESO-1, CDK-4, β-catenin, MUM-1, Caspase-8, KIAA0205, HPVE, SART-1, PRAME, p15, melanoma tumor antigens (Kawakami et al. (1994) Proc. Natl. Acad. Sci. USA 91:3515; Kawakami et al. (1994) J. Exp. Med., 180:347; Kawakami et al. (1994) Cancer Res. 54:3124), MART-1, gp100 MAGE-1, MAGE-2, MAGE-3, CEA, TRP-1, TRP-2, P-15, tyrosinase (Brichard et al. (1993) J. Exp. Med. 178:489); HER-2/neu gene product (U.S. Pat. No. 4,968,603), CA125, LK26, FB5 (endosialin), TAG 72, AFP, CA19-9, NSE, DU-PAN-2, CA50, SPan-1, CA72-4, HCG, STN (sialyl Tn antigen), c-erbB-2 proteins, PSA, L-CanAg, estrogen receptor, milk fat globulin, p53 tumor suppressor protein (Levine, (1993) Ann. Rev. Biochem. 62:623); mucin antigens (PCT Publication No. WO 90/05142); telomerases; nuclear matrix proteins; prostatic acid phosphatase; papilloma virus antigens; and/or antigens now known or later discovered to be associated with the following cancers: melanoma, adenocarcinoma, thymoma, lymphoma (e.g., non-Hodgkin's lymphoma, Hodgkin's lymphoma), sarcoma, lung cancer, liver cancer, colon cancer, leukemia, uterine cancer, breast cancer, prostate cancer, ovarian cancer, cervical cancer, bladder cancer, kidney cancer, pancreatic cancer, brain cancer and any other cancer or malignant condition now known or later identified (see, e.g., Rosenberg, (1996) Ann. Rev. Med. 47:481-91).

As a further alternative, the heterologous nucleic acid can encode any polypeptide that is desirably produced in a cell in vitro, ex vivo, or in vivo. For example, the virus vectors may be introduced into cultured cells and the expressed nucleic acid product isolated therefrom.

It will be understood by those skilled in the art that the heterologous nucleic acid(s) of interest can be operably associated with appropriate control sequences. For example, the heterologous nucleic acid can be operably associated with expression control elements, such as transcription/translation control signals, origins of replication, polyadenylation signals, internal ribosome entry sites (IRES), promoters, and/or enhancers, and the like.

Further, regulated expression of the heterologous nucleic acid(s) of interest can be achieved at the post-transcriptional level, e.g., by regulating selective splicing of different introns by the presence or absence of an oligonucleotide, small molecule and/or other compound that selectively blocks splicing activity at specific sites (e.g., as described in PCT Publication No. WO 2006/119137).

Those skilled in the art will appreciate that a variety of promoter/enhancer elements can be used depending on the level and tissue-specific expression desired. The promoter/enhancer can be constitutive or inducible, depending on the pattern of expression desired. The promoter/enhancer can be native or foreign and can be a natural or a synthetic sequence. By foreign, it is intended that the transcriptional initiation region is not found in the wild-type host into which the transcriptional initiation region is introduced.

In particular embodiments, the promoter/enhancer elements can be native to the target cell or subject to be treated. In representative embodiments, the promoters/enhancer element can be native to the heterologous nucleic acid sequence. The promoter/enhancer element is generally chosen so that it functions in the target cell(s) of interest. Further, in particular embodiments the promoter/enhancer element is a mammalian promoter/enhancer element. The promoter/enhancer element may be constitutive or inducible.

Inducible expression control elements are typically advantageous in those applications in which it is desirable to provide regulation over expression of the heterologous nucleic acid sequence(s). Inducible promoters/enhancer elements for gene delivery can be tissue-specific or preferred promoter/enhancer elements, and include muscle specific or preferred (including cardiac, skeletal and/or smooth muscle specific or preferred), neural tissue specific or preferred (including brain-specific or preferred), eye specific or preferred (including retina-specific and cornea-specific), liver specific or preferred, bone marrow specific or preferred, pancreatic specific or preferred, spleen specific or preferred, and/or lung specific or preferred promoter/enhancer elements. Other inducible promoter/enhancer elements include hormone-inducible and metal-inducible elements. Exemplary inducible promoters/enhancer elements include, but are not limited to, a Tet on/off element, a RU486-inducible promoter, an ecdysone-inducible promoter, a rapamycin-inducible promoter, and a metallothionein promoter.

In embodiments wherein the heterologous nucleic acid sequence(s) is transcribed and then translated in the target cells, specific initiation signals are generally included for efficient translation of inserted protein coding sequences. These exogenous translational control sequences, which may include the ATG initiation codon and adjacent sequences, can be of a variety of origins, both natural and synthetic.

The virus vectors according to the present invention provide a means for delivering heterologous nucleic acids into a broad range of cells, including dividing and non-dividing cells. The virus vectors can be employed, for example, to deliver a nucleic acid of interest to a cell in vitro, e.g., to produce a polypeptide in vitro or for ex vivo gene therapy. The virus vectors are additionally useful in a method of delivering a nucleic acid to a subject in need thereof, e.g., to express an immunogenic and/or therapeutic polypeptide and/or a functional RNA. In this manner, the polypeptide and/or functional RNA can be produced in vivo in the subject. The subject can be in need of the polypeptide because the subject has a deficiency of the polypeptide. Further, the method can be practiced because the production of the polypeptide and/or functional RNA in the subject may impart some beneficial effect.

The virus vectors can also be used to produce a polypeptide of interest and/or functional RNA in cultured cells or in a subject (e.g., using the subject as a bioreactor to produce the polypeptide and/or to observe the effects of the functional RNA on the subject, for example, in connection with screening methods).

In general, the virus vectors of the present invention can be employed to deliver a heterologous nucleic acid encoding a polypeptide and/or functional RNA (e.g., a therapeutic polypeptide, e.g., a therapeutic nucleic acid) to treat and/or prevent any disease state or disorder for which it is beneficial to deliver a therapeutic polypeptide and/or functional RNA, e.g., to a subject in need thereof, e.g., wherein the subject has or is at risk for a disease state or disorder. In some embodiments, the disease state is a CNS disease or disorder. In some embodiments, the subject has or is at risk of having pain associated with a disease or disorder. In some embodiments, the subject is a human. In some embodiments, the subject is in utero.

Illustrative disease states include, but are not limited to: cystic fibrosis (cystic fibrosis transmembrane regulator protein) and other diseases of the lung, hemophilia A (Factor VIII), hemophilia B (Factor IX), thalassemia (β-globin), anemia (erythropoietin) and other blood disorders, Alzheimer's disease (GDF; neprilysin), multiple sclerosis (β-interferon), Parkinson's disease (glial-cell line derived neurotrophic factor [GDNF]), Huntington's disease (RNAi to remove repeats), amyotrophic lateral sclerosis, epilepsy (galanin, neurotrophic factors), and other neurological disorders, cancer (endostatin, angiostatin, TRAIL, FAS-ligand, cytokines including interferons; RNAi including RNAi against VEGF or the multiple drug resistance gene product, mir-26a [e.g., for hepatocellular carcinoma]), diabetes mellitus (insulin), muscular dystrophies including Duchenne (dystrophin, mini-dystrophin, insulin-like growth factor I, a sarcoglycan [e.g., α, β, γ], RNAi against myostatin, myostatin propeptide, follistatin, activin type II soluble receptor, anti-inflammatory polypeptides such as the Ikappa B dominant mutant, sarcospan, utrophin, mini-utrophin, antisense or RNAi against splice junctions in the dystrophin gene to induce exon skipping [see e.g., PCT Publication No. WO/2003/095647], antisense against U7 snRNAs to induce exon skipping [see e.g., PCT Publication No. WO/2006/021724], and antibodies or antibody fragments against myostatin or myostatin propeptide) and Becker, Gaucher disease (glucocerebrosidase), Hurler's disease (α-L-iduronidase), adenosine deaminase deficiency (adenosine deaminase), glycogen storage diseases (e.g., Fabry disease [α-galactosidase] and Pompe disease [lysosomal acid α-glucosidase]) and other metabolic disorders, congenital emphysema (al-antitrypsin), Lesch-Nyhan Syndrome (hypoxanthine guanine phosphoribosyl transferase), Niemann-Pick disease (sphingomyelinase), Tays Sachs disease (lysosomal hexosaminidase A), Maple Syrup Urine Disease (branched-chain keto acid dehydrogenase), retinal degenerative diseases (and other diseases of the eye and retina; e.g., PDGF for macular degeneration and/or vasohibin or other inhibitors of VEGF or other angiogenesis inhibitors to treat/prevent retinal disorders, e.g., in Type I diabetes), diseases of solid organs such as brain (including Parkinson's Disease [GDNF], astrocytomas [endostatin, angiostatin and/or RNAi against VEGF], glioblastomas [endostatin, angiostatin and/or RNAi against VEGF]), liver, kidney, heart including congestive heart failure or peripheral artery disease (PAD) (e.g., by delivering protein phosphatase inhibitor I (I-1) and fragments thereof (e.g., I1C), serca2a, zinc finger proteins that regulate the phospholamban gene, Barkct, β2-adrenergic receptor, β2-adrenergic receptor kinase (BARK), phosphoinositide-3 kinase (PI3 kinase), S100A1, parvalbumin, adenylyl cyclase type 6, a molecule that effects G-protein coupled receptor kinase type 2 knockdown such as a truncated constitutively active bARKct; calsarcin, RNAi against phospholamban; phospholamban inhibitory or dominant-negative molecules such as phospholamban S16E, etc.), arthritis (insulin-like growth factors), joint disorders (insulin-like growth factor 1 and/or 2), intimal hyperplasia (e.g., by delivering enos, inos), improve survival of heart transplants (superoxide dismutase), AIDS (soluble CD4), muscle wasting (insulin-like growth factor I), kidney deficiency (erythropoietin), anemia (erythropoietin), arthritis (anti-inflammatory factors such as IRAP and TNFα soluble receptor), hepatitis (α-interferon), LDL receptor deficiency (LDL receptor), hyperammonemia (ornithine transcarbamylase), Krabbe's disease (galactocerebrosidase), Batten's disease, spinal cerebral ataxias including SCA1, SCA2 and SCA3, phenylketonuria (phenylalanine hydroxylase), autoimmune diseases, congenital neurodegenerative disorders (e.g., monogenic neurodegenerative disorders) such as mucopolysaccharidosis (including, but not limited to, Mucopolysaccharidosis Type I (also known as Hurler syndrome, Hurler-Scheie Syndrome, or Scheie syndrome, IDUA, alpha-L-iduronidase), Mucopolysaccharidosis Type II (also known as Hunter syndrome, IDS, I2L enzyme), Mucopolysaccharidosis Type III (also known as Sanfilippo syndrome, GNS [N-acetylglucosamine-6-sulfatase], HGSNAT [heparan-alpha-glucosaminide N-acetyltransferase], NAGLU [alpha-N-acetylglucosaminidase], and/or SGSH [sulfamidase]), Mucopolysaccharidosis Type IV (also known as Morquio syndrome, GALNS [galatosamine (N-acetyl)-6-sulfatase] and/or GLB1 [beta-galactosidase]), Mucopolysaccharidosis Type V (also known as Scheie syndrome, now a subgroup of type I, also IDUA, alpha-L-iduronidase), Mucopolysaccharidosis Type VI (also known as Maroteaux-Lamy syndrome, ARSB, arylsulfatase B), Mucopolysaccharidosis Type VII (also known as Sly syndrome, GUSB, beta-glucuronidase), Mucopolysaccharidosis Type IX (also known as Natowicz syndrome, HYAL1, hyaluronidase) and/or leukodystrophy (including, but not limited to, adult-onset autosomal dominant leukodystrophy (ADLD; LMNB1, lamin B1), Aicardi-Goutieres syndrome (TREX1, RNASEHSB, RNASEH2C, and/or RNASEH2A), Alexander disease (FRAP, glial fibrillary acidic protein), CADASIL (Notch3), Canavan disease (ASPA, aspartoacylase), CARASIL (HTRA1, serine protease HTRA1), cerebrotendinous xanthomatosis (“CTX,” CYP27A1, sterol 27-hydroxylase) childhood ataxia and cerebral hypomyelination (CACH)/vanishing white matter disease (VWMD) (eIF2B, eukaryotic initiation factor 2B), Fabry disease (GLA, alpha-galactosidase A), fucosidosis (FUCA1, alpha-L-fucosidase), GM1 gangliosidosis (GLB1, beta-galactosidase), L-2-hydroxyglutaric aciduria (L2HDGH, L-2-hydroxyglutarate dehydrogenase), Krabbe disease (GALC, galactocerebrosidase), megalencephalic leukoencephalopathy with subcortical cysts (“MLC,” MLC1 and/or HEPACAM), metachromatic leukodystrophy (ASA, arylsulphatase A), multiple sulfatase deficiency (“MSD,” SUMF1, sulfatase modifying factor 1 affecting all sulfatase enzymes), Pelizaeus-Merzbacher disease (also known as “X-linked spastic paraplegia,” PLP1 [X-linked proteolipid protein 1] and/or GJA12 [gap junction protein 12]), Pol III-Related Leukodystrophies (POLR3A and/or POLR3B), Refsum disease (PHYH, [phytanoyl-CoA hydroxylase] and/or Pex7 [PHYH importer into peroxisomes]), salla disease (also known as “free sialic acid storage disease,” SLC17A5, sialic acid transporter), Sjogren-Larsson syndrome (ÁLDH3A2, aldehyde dehydrogenase), X-linked adrenoleukodystrophy (“ALD,” ABCD1, peroxisomal ATPase Binding Cassette protein), Zellweger syndrome spectrum disorders (also known as peroxisomal biogenesis disorders, PEX1, PEX2, PEX3, PEX4, PEX5, PEX10, PEX11B, PEX12, PEX13, PEX14, PEX16, PEX19, PEX26), and the like. The invention can further be used following organ transplantation to increase the success of the transplant and/or to reduce the negative side effects of organ transplantation or adjunct therapies (e.g., by administering immunosuppressant agents or inhibitory nucleic acids to block cytokine production). As another example, bone morphogenic proteins (including BNP 2, 7, etc., RANKL and/or VEGF) can be administered with a bone allograft, for example, following a break or surgical removal in a cancer patient.

Thus, in some embodiments, the present invention provides a method of treating a disease in a subject in need thereof, comprising introducing a therapeutic nucleic acid into a cell of the subject by administering to the subject the virus vector and/or composition of the present invention, under conditions whereby the therapeutic nucleic acid is expressed in the subject.

The invention can also be used to produce induced pluripotent stem cells (iPS). For example, a virus vector of the invention can be used to deliver stem cell associated nucleic acid(s) into a non-pluripotent cell, such as adult fibroblasts, skin cells, liver cells, renal cells, adipose cells, cardiac cells, neural cells, epithelial cells, endothelial cells, and the like. Nucleic acids encoding factors associated with stem cells are known in the art. Nonlimiting examples of such factors associated with stem cells and pluripotency include Oct-3/4, the SOX family (e.g., SOX1, SOX2, SOX3 and/or SOX15), the Klf family (e.g., Klf1, Klf2, Klf4 and/or Klf5), the Myc family (e.g., C-myc, L-myc and/or N-myc), NANOG and/or LIN28.

The invention can also be practiced to treat and/or prevent a metabolic disorder such as diabetes (e.g., insulin), hemophilia (e.g., Factor IX or Factor VIII), a lysosomal storage disorder such as a mucopolysaccharidosis disorder (e.g., Sly syndrome [β-glucuronidase], Hurler Syndrome [α-L-iduronidase], Scheie Syndrome [α-L-iduronidase], Hurler-Scheie Syndrome [α-L-iduronidase], Hunter's Syndrome [iduronate sulfatase], Sanfilippo Syndrome A [heparan sulfamidase], B [N-acetylglucosaminidase], C [acetyl-CoA:α-glucosaminide acetyltransferase], D [N-acetylglucosamine 6-sulfatase], Morquio Syndrome A [galactose-6-sulfate sulfatase], B [β-galactosidase], Maroteaux-Lamy Syndrome [N-acetylgalactosamine-4-sulfatase], etc.), Fabry disease (α-galactosidase), Gaucher's disease (glucocerebrosidase), or a glycogen storage disorder (e.g., Pompe disease; lysosomal acid α-glucosidase).

Gene transfer has substantial potential use for understanding and providing therapy for disease states. There are a number of inherited diseases in which defective genes are known and have been cloned. In general, the above disease states fall into two classes: deficiency states, usually of enzymes, which are generally inherited in a recessive manner, and unbalanced states, which may involve regulatory or structural proteins, and which are typically inherited in a dominant manner. For deficiency state diseases, gene transfer can be used to bring a normal gene into affected tissues for replacement therapy, as well as to create animal models for the disease using antisense mutations. For unbalanced disease states, gene transfer can be used to create a disease state in a model system, which can then be used in efforts to counteract the disease state. Thus, virus vectors according to the present invention permit the treatment and/or prevention of genetic diseases.

The virus vectors according to the present invention may also be employed to provide a functional RNA to a cell in vitro or in vivo. Expression of the functional RNA in the cell, for example, can diminish expression of a particular target protein by the cell. Accordingly, functional RNA can be administered to decrease expression of a particular protein in a subject in need thereof. Functional RNA can also be administered to cells in vitro to regulate gene expression and/or cell physiology, e.g., to optimize cell or tissue culture systems or in screening methods.

In addition, virus vectors according to the instant invention find use in diagnostic and screening methods, whereby a nucleic acid of interest is transiently or stably expressed in a cell culture system, or alternatively, a transgenic animal model.

The virus vectors of the present invention can also be used for various non-therapeutic purposes, including but not limited to use in protocols to assess gene targeting, clearance, transcription, translation, etc., as would be apparent to one skilled in the art. The virus vectors can also be used for the purpose of evaluating safety (spread, toxicity, immunogenicity, etc.). Such data, for example, are considered by the United States Food and Drug Administration as part of the regulatory approval process prior to evaluation of clinical efficacy.

As a further aspect, the virus vectors of the present invention may be used to produce an immune response in a subject. According to this embodiment, a virus vector comprising a heterologous nucleic acid sequence encoding an immunogenic polypeptide can be administered to a subject, and an active immune response is mounted by the subject against the immunogenic polypeptide. Immunogenic polypeptides are as described hereinabove. In some embodiments, a protective immune response is elicited.

Alternatively, the virus vector may be administered to a cell ex vivo and the altered cell is administered to the subject. The virus vector comprising the heterologous nucleic acid is introduced into the cell, and the cell is administered to the subject, where the heterologous nucleic acid encoding the immunogen can be expressed and induce an immune response in the subject against the immunogen. In particular embodiments, the cell is an antigen-presenting cell (e.g., a dendritic cell).

An “active immune response” or “active immunity” is characterized by “participation of host tissues and cells after an encounter with the immunogen. It involves differentiation and proliferation of immunocompetent cells in lymphoreticular tissues, which lead to synthesis of antibody or the development of cell-mediated reactivity, or both.” Herbert B. Herscowitz, Immunophysiology: Cell Function and Cellular Interactions in Antibody Formation, in IMMUNOLOGY: BASIC PROCESSES 117 (Joseph A. Bellanti ed., 1985). Alternatively stated, an active immune response is mounted by the host after exposure to an immunogen by infection or by vaccination. Active immunity can be contrasted with passive immunity, which is acquired through the “transfer of preformed substances (antibody, transfer factor, thymic graft, interleukin-2) from an actively immunized host to a non-immune host.” Id.

A “protective” immune response or “protective” immunity as used herein indicates that the immune response confers some benefit to the subject in that it prevents or reduces the incidence of disease. Alternatively, a protective immune response or protective immunity may be useful in the treatment and/or prevention of disease, in particular cancer or tumors (e.g., by preventing cancer or tumor formation, by causing regression of a cancer or tumor and/or by preventing metastasis and/or by preventing growth of metastatic nodules). The protective effects may be complete or partial, as long as the benefits of the treatment outweigh any disadvantages thereof.

In particular embodiments, the virus vector or cell comprising the heterologous nucleic acid can be administered in an immunogenically effective amount, as described herein.

The virus vectors of the present invention can also be administered for cancer immunotherapy by administration of a virus vector expressing one or more cancer cell antigens (or an immunologically similar molecule) or any other immunogen that produces an immune response against a cancer cell. To illustrate, an immune response can be produced against a cancer cell antigen in a subject by administering a virus vector comprising a heterologous nucleic acid encoding the cancer cell antigen, for example to treat a patient with cancer and/or to prevent cancer from developing in the subject. The virus vector may be administered to a subject in vivo or by using ex vivo methods, as described herein. Alternatively, the cancer antigen can be expressed as part of the virus capsid or be otherwise associated with the virus capsid (e.g., as described above).

As another alternative, any other therapeutic nucleic acid (e.g., RNAi) or polypeptide (e.g., cytokine) known in the art can be administered to treat and/or prevent cancer.

As used herein, the term “cancer” encompasses tumor-forming cancers. Likewise, the term “cancerous tissue” encompasses tumors. A “cancer cell antigen” encompasses tumor antigens.

The term “cancer” has its understood meaning in the art, for example, an uncontrolled growth of tissue that has the potential to spread to distant sites of the body (i.e., metastasize). Exemplary cancers include, but are not limited to melanoma, adenocarcinoma, thymoma, lymphoma (e.g., non-Hodgkin's lymphoma, Hodgkin's lymphoma), sarcoma, lung cancer, liver cancer, colon cancer, leukemia, uterine cancer, breast cancer, prostate cancer, ovarian cancer, cervical cancer, bladder cancer, kidney cancer, pancreatic cancer, brain cancer and any other cancer or malignant condition now known or later identified. In representative embodiments, the invention provides a method of treating and/or preventing tumor-forming cancers.

The term “tumor” is also understood in the art, for example, as an abnormal mass of undifferentiated cells within a multicellular organism. Tumors can be malignant or benign. In representative embodiments, the methods disclosed herein are used to prevent and treat malignant tumors.

By the terms “treating cancer,” “treatment of cancer” and equivalent terms it is intended that the severity of the cancer is reduced or at least partially eliminated and/or the progression of the disease is slowed and/or controlled and/or the disease is stabilized. In particular embodiments, these terms indicate that metastasis of the cancer is prevented or reduced or at least partially eliminated and/or that growth of metastatic nodules is prevented or reduced or at least partially eliminated.

By the terms “prevention of cancer” or “preventing cancer” and equivalent terms it is intended that the methods at least partially eliminate or reduce and/or delay the incidence and/or severity of the onset of cancer. Alternatively stated, the onset of cancer in the subject may be reduced in likelihood or probability and/or delayed.

In particular embodiments, cells may be removed from a subject with cancer and contacted with a virus vector expressing a cancer cell antigen according to the instant invention. The modified cell is then administered to the subject, whereby an immune response against the cancer cell antigen is elicited. This method can be advantageously employed with immunocompromised subjects that cannot mount a sufficient immune response in vivo (i.e., cannot produce enhancing antibodies in sufficient quantities).

It is known in the art that immune responses may be enhanced by immunomodulatory cytokines (e.g., α-interferon, β-interferon, γ-interferon, ω-interferon, τ-interferon, interleukin-1α, interleukin-1β, interleukin-2, interleukin-3, interleukin-4, interleukin 5, interleukin-6, interleukin-7, interleukin-8, interleukin-9, interleukin-10, interleukin-11, interleukin 12, interleukin-13, interleukin-14, interleukin-18, B cell Growth factor, CD40 Ligand, tumor necrosis factor-α, tumor necrosis factor-β, monocyte chemoattractant protein-1, granulocyte-macrophage colony stimulating factor, and lymphotoxin). Accordingly, immunomodulatory cytokines (preferably, CTL inductive cytokines) may be administered to a subject in conjunction with the virus vector.

Cytokines may be administered by any method known in the art. Exogenous cytokines may be administered to the subject, or alternatively, a nucleic acid encoding a cytokine may be delivered to the subject using a suitable vector, and the cytokine produced in vivo.

### Subjects, Pharmaceutical Formulations, and Modes of Administration

Virus vectors and capsids according to the present invention find use in both veterinary and medical applications. Suitable subjects include both avians and mammals. The term “avian” as used herein includes, but is not limited to, chickens, ducks, geese, quail, turkeys, pheasant, parrots, parakeets, and the like. The term “mammal” as used herein includes, but is not limited to, humans, non-human primates, bovines, ovines, caprines, equines, felines, canines, lagomorphs, etc. Human subjects include in utero (e.g., embryos, fetuses), neonates, infants, juveniles, adults and geriatric subjects.

In representative embodiments, the subject is “in need of” the methods of the invention and thus in some embodiments can be a “subject in need thereof.”

In particular embodiments, the present invention provides a pharmaceutical composition comprising a virus vector and/or capsid of the invention in a pharmaceutically acceptable carrier and, optionally, other medicinal agents, pharmaceutical agents, stabilizing agents, buffers, carriers, adjuvants, diluents, etc. For injection, the carrier will typically be a liquid. For other methods of administration, the carrier may be either solid or liquid. For inhalation administration, the carrier will be respirable, and optionally can be in solid or liquid particulate form.

By “pharmaceutically acceptable” it is meant a material that is not toxic or otherwise undesirable, i.e., the material may be administered to a subject without causing any undesirable biological effects.

One aspect of the present invention is a method of transferring a nucleic acid to a cell in vitro. The virus vector may be introduced into the cells at the appropriate multiplicity of infection according to standard transduction methods suitable for the particular target cells. Titers of virus vector to administer can vary, depending upon the target cell type and number, and the particular virus vector, and can be determined by those of skill in the art without undue experimentation. In representative embodiments, at least about 10³ infectious units, optionally at least about 10⁵ infectious units are introduced to the cell.

The cell(s) into which the virus vector is introduced can be of any type, including but not limited to neural cells (including cells of the peripheral and central nervous systems, in particular, brain cells such as neurons and oligodendrocytes), lung cells, cells of the eye (including retinal cells, retinal pigment epithelium, and corneal cells), epithelial cells (e.g., gut and respiratory epithelial cells), muscle cells (e.g., skeletal muscle cells, cardiac muscle cells, smooth muscle cells and/or diaphragm muscle cells), dendritic cells, pancreatic cells (including islet cells), hepatic cells, myocardial cells, bone cells (e.g., bone marrow stem cells), hematopoietic stem cells, spleen cells, keratinocytes, fibroblasts, endothelial cells, prostate cells, germ cells, and the like. In representative embodiments, the cell can be any progenitor cell. As a further embodiment, the cell can be a stem cell (e.g., neural stem cell, liver stem cell). As still a further embodiment, the cell can be a cancer or tumor cell. Moreover, the cell can be from any species of origin, as indicated above.

The virus vector can be introduced into cells in vitro for the purpose of administering the modified cell to a subject. In particular embodiments, the cells have been removed from a subject, the virus vector is introduced therein, and the cells are then administered back into the subject. Methods of removing cells from subject for manipulation ex vivo, followed by introduction back into the subject are known in the art (see, e.g., U.S. Pat. No. 5,399,346). Alternatively, the recombinant virus vector can be introduced into cells from a donor subject, into cultured cells, or into cells from any other suitable source, and the cells are administered to a subject in need thereof (i.e., a “recipient” subject).

Suitable cells for ex vivo nucleic acid delivery are as described above. Dosages of the cells to administer to a subject will vary upon the age, condition and species of the subject, the type of cell, the nucleic acid being expressed by the cell, the mode of administration, and the like. Typically, at least about 10² to about 10⁸ cells or at least about 10³ to about 10⁶ cells will be administered per dose in a pharmaceutically acceptable carrier. In particular embodiments, the cells transduced with the virus vector are administered to the subject in a treatment effective or prevention effective amount in combination with a pharmaceutical carrier.

In some embodiments, the virus vector is introduced into a cell and the cell can be administered to a subject to elicit an immunogenic response against the delivered polypeptide (e.g., expressed as a transgene or in the capsid). Typically, a quantity of cells expressing an immunogenically effective amount of the polypeptide in combination with a pharmaceutically acceptable carrier is administered. An “immunogenically effective amount” is an amount of the expressed polypeptide that is sufficient to evoke an active immune response against the polypeptide in the subject to which the pharmaceutical formulation is administered. In particular embodiments, the dosage is sufficient to produce a protective immune response (as defined above). The degree of protection conferred need not be complete or permanent, as long as the benefits of administering the immunogenic polypeptide outweigh any disadvantages thereof.

In some embodiments, the subject may have a reduced immunologic profile (e.g., immunologic response, e.g., antigenic cross reactivity) when contacted with a virus vector of the present invention as compared to a control, e.g., when contacted with another AAV virus vector (e.g., any AAV serotype listed in Table 1).

A further aspect of the invention is a method of administering the virus vector and/or virus capsid to a subject. Administration of the virus vectors and/or capsids according to the present invention to a human subject or an animal in need thereof can be by any means known in the art. Optionally, the virus vector and/or capsid can be delivered in a treatment effective or prevention effective dose in a pharmaceutically acceptable carrier.

The virus vectors and/or capsids of the invention can further be administered to elicit an immunogenic response (e.g., as a vaccine). Typically, immunogenic compositions of the present invention comprise an immunogenically effective amount of virus vector and/or capsid in combination with a pharmaceutically acceptable carrier. Optionally, the dosage is sufficient to produce a protective immune response (as defined above).

Dosages of the virus vector and/or capsid to be administered to a subject depend upon the mode of administration, the disease or condition to be treated and/or prevented, the individual subject's condition, the particular virus vector or capsid, the nucleic acid to be delivered, and the like, and can be determined in a routine manner. Exemplary doses for achieving therapeutic effects are titers of at least about 10⁵, 10⁶, 10⁷, 10⁸, 10⁹, 10¹⁰, 10¹¹, 10¹², 10³, 10¹⁴, 10¹⁵ transducing units, optionally about 10⁸-10¹³ transducing units.

In particular embodiments, more than one administration (e.g., two, three, four or more administrations) may be employed to achieve the desired level of gene expression over a period of various intervals, e.g., daily, weekly, monthly, yearly, etc.

Exemplary modes of administration include oral, rectal, transmucosal, intranasal, inhalation (e.g., via an aerosol), buccal (e.g., sublingual), vaginal, intrathecal, intraocular, transdermal, in utero (or in ovo), parenteral (e.g., intravenous, subcutaneous, intradermal, intramuscular [including administration to skeletal, diaphragm and/or cardiac muscle], intradermal, intrapleural, intracerebral, and intraarticular), topical (e.g., to both skin and mucosal surfaces, including airway surfaces, and transdermal administration), intralymphatic, and the like, as well as direct tissue or organ injection (e.g., to liver, skeletal muscle, cardiac muscle, diaphragm muscle or brain). Administration can also be to a tumor (e.g., in or near a tumor or a lymph node). The most suitable route in any given case will depend on the nature and severity of the condition being treated and/or prevented and on the nature of the particular vector that is being used.

Administration to skeletal muscle according to the present invention includes but is not limited to administration to skeletal muscle in the limbs (e.g., upper arm, lower arm, upper leg, and/or lower leg), back, neck, head (e.g., tongue), thorax, abdomen, pelvis/perineum, and/or digits. Suitable skeletal muscles include but are not limited to abductor digiti minimi (in the hand), abductor digiti minimi (in the foot), abductor hallucis, abductor ossis metatarsi quinti, abductor pollicis brevis, abductor pollicis longus, adductor brevis, adductor hallucis, adductor longus, adductor magnus, adductor pollicis, anconeus, anterior scalene, articularis genus, biceps brachii, biceps femoris, brachialis, brachioradialis, buccinator, coracobrachialis, corrugator supercilii, deltoid, depressor anguli oris, depressor labii inferioris, digastric, dorsal interossei (in the hand), dorsal interossei (in the foot), extensor carpi radialis brevis, extensor carpi radialis longus, extensor carpi ulnaris, extensor digiti minimi, extensor digitorum, extensor digitorum brevis, extensor digitorum longus, extensor hallucis brevis, extensor hallucis longus, extensor indicis, extensor pollicis brevis, extensor pollicis longus, flexor carpi radialis, flexor carpi ulnaris, flexor digiti minimi brevis (in the hand), flexor digiti minimi brevis (in the foot), flexor digitorum brevis, flexor digitorum longus, flexor digitorum profundus, flexor digitorum superficialis, flexor hallucis brevis, flexor hallucis longus, flexor pollicis brevis, flexor pollicis longus, frontalis, gastrocnemius, geniohyoid, gluteus maximus, gluteus medius, gluteus minimus, gracilis, iliocostalis cervicis, iliocostalis lumborum, iliocostalis thoracis, illiacus, inferior gemellus, inferior oblique, inferior rectus, infraspinatus, interspinalis, intertransversi, lateral pterygoid, lateral rectus, latissimus dorsi, levator anguli oris, levator labii superioris, levator labii superioris alaeque nasi, levator palpebrae superioris, levator scapulae, long rotators, longissimus capitis, longissimus cervicis, longissimus thoracis, longus capitis, longus colli, lumbricals (in the hand), lumbricals (in the foot), masseter, medial pterygoid, medial rectus, middle scalene, multifidus, mylohyoid, obliquus capitis inferior, obliquus capitis superior, obturator externus, obturator internus, occipitalis, omohyoid, opponens digiti minimi, opponens pollicis, orbicularis oculi, orbicularis oris, palmar interossei, palmaris brevis, palmaris longus, pectineus, pectoralis major, pectoralis minor, peroneus brevis, peroneus longus, peroneus tertius, piriformis, plantar interossei, plantaris, platysma, popliteus, posterior scalene, pronator quadratus, pronator teres, psoas major, quadratus femoris, quadratus plantae, rectus capitis anterior, rectus capitis lateralis, rectus capitis posterior major, rectus capitis posterior minor, rectus femoris, rhomboid major, rhomboid minor, risorius, sartorius, scalenus minimus, semimembranosus, semispinalis capitis, semispinalis cervicis, semispinalis thoracis, semitendinosus, serratus anterior, short rotators, soleus, spinalis capitis, spinalis cervicis, spinalis thoracis, splenius capitis, splenius cervicis, sternocleidomastoid, sternohyoid, sternothyroid, stylohyoid, subclavius, subscapularis, superior gemellus, superior oblique, superior rectus, supinator, supraspinatus, temporalis, tensor fascia lata, teres major, teres minor, thoracis, thyrohyoid, tibialis anterior, tibialis posterior, trapezius, triceps brachii, vastus intermedius, vastus lateralis, vastus medialis, zygomaticus major, and zygomaticus minor, and any other suitable skeletal muscle as known in the art.

The virus vector and/or capsid can be delivered to skeletal muscle by intravenous administration, intra-arterial administration, intraperitoneal administration, limb perfusion, (optionally, isolated limb perfusion of a leg and/or arm; see e.g. Arruda et al. (2005) Blood 105:3458-3464), and/or direct intramuscular injection. In particular embodiments, the virus vector and/or capsid is administered to a limb (arm and/or leg) of a subject (e.g., a subject with muscular dystrophy such as DMD) by limb perfusion, optionally isolated limb perfusion (e.g., by intravenous or intra-articular administration). In embodiments of the invention, the virus vectors and/or capsids of the invention can advantageously be administered without employing “hydrodynamic” techniques. Tissue delivery (e.g., to muscle) of vectors is often enhanced by hydrodynamic techniques (e.g., intravenous/intravenous administration in a large volume), which increase pressure in the vasculature and facilitate the ability of the vector to cross the endothelial cell barrier. In particular embodiments, the viral vectors and/or capsids of the invention can be administered in the absence of hydrodynamic techniques such as high volume infusions and/or elevated intravascular pressure (e.g., greater than normal systolic pressure, for example, less than or equal to a 5%, 10%, 15%, 20%, 25% increase in intravascular pressure over normal systolic pressure). Such methods may reduce or avoid the side effects associated with hydrodynamic techniques such as edema, nerve damage and/or compartment syndrome.

Administration to cardiac muscle includes administration to the left atrium, right atrium, left ventricle, right ventricle and/or septum. The virus vector and/or capsid can be delivered to cardiac muscle by intravenous administration, intra-arterial administration such as intra-aortic administration, direct cardiac injection (e.g., into left atrium, right atrium, left ventricle, right ventricle), and/or coronary artery perfusion.

Administration to diaphragm muscle can be by any suitable method including intravenous administration, intra-arterial administration, and/or intra-peritoneal administration.

Delivery to a target tissue can also be achieved by delivering a depot comprising the virus vector and/or capsid. In representative embodiments, a depot comprising the virus vector and/or capsid is implanted into skeletal, cardiac and/or diaphragm muscle tissue or the tissue can be contacted with a film or other matrix comprising the virus vector and/or capsid. Such implantable matrices or substrates are described, e.g., in U.S. Pat. No. 7,201,898.

In particular embodiments, a virus vector and/or virus capsid according to the present invention is administered to skeletal muscle, diaphragm muscle and/or cardiac muscle (e.g., to treat and/or prevent muscular dystrophy, heart disease [for example, PAD or congestive heart failure]).

In representative embodiments, the invention is used to treat and/or prevent disorders of skeletal, cardiac and/or diaphragm muscle.

In a representative embodiment, the invention provides a method of treating and/or preventing muscular dystrophy in a subject in need thereof, the method comprising: administering a treatment or prevention effective amount of a virus vector of the invention to a mammalian subject, wherein the virus vector comprises a heterologous nucleic acid encoding dystrophin, a mini-dystrophin, a micro-dystrophin, myostatin propeptide, follistatin, activin type II soluble receptor, IGF-1, anti-inflammatory polypeptides such as the Ikappa B dominant mutant, sarcospan, utrophin, a micro-dystrophin, laminin-α2, α-sarcoglycan, β-sarcoglycan, γ-sarcoglycan, δ-sarcoglycan, IGF-1, an antibody or antibody fragment against myostatin or myostatin propeptide, and/or RNAi against myostatin. In particular embodiments, the virus vector can be administered to skeletal, diaphragm and/or cardiac muscle as described elsewhere herein.

Alternatively, the invention can be practiced to deliver a nucleic acid to skeletal, cardiac or diaphragm muscle, which is used as a platform for production of a polypeptide (e.g., an enzyme) or functional RNA (e.g., RNAi, microRNA, antisense RNA) that normally circulates in the blood or for systemic delivery to other tissues to treat and/or prevent a disorder (e.g., a metabolic disorder, such as diabetes [e.g., insulin], hemophilia [e.g., Factor IX or Factor VIII], a mucopolysaccharide disorder [e.g., Sly syndrome, Hurler Syndrome, Scheie Syndrome, Hurler-Scheie Syndrome, Hunter's Syndrome, Sanfilippo Syndrome A, B, C, D, Morquio Syndrome, Maroteaux-Lamy Syndrome, etc.] or a lysosomal storage disorder such as Gaucher's disease [glucocerebrosidase] or Fabry disease [α-galactosidase A] or a glycogen storage disorder such as Pompe disease [lysosomal acid α glucosidase]). Other suitable proteins for treating and/or preventing metabolic disorders are described herein. The use of muscle as a platform to express a nucleic acid of interest is described in U.S. Patent Publication No. 20020192189.

Thus, as one aspect, the invention further encompasses a method of treating and/or preventing a metabolic disorder in a subject in need thereof, the method comprising: administering a treatment or prevention effective amount of a virus vector of the invention to skeletal muscle of a subject, wherein the virus vector comprises a heterologous nucleic acid encoding a polypeptide, wherein the metabolic disorder is a result of a deficiency and/or defect in the polypeptide. Illustrative metabolic disorders and heterologous nucleic acids encoding polypeptides are described herein. Optionally, the polypeptide is secreted (e.g., a polypeptide that is a secreted polypeptide in its native state or that has been engineered to be secreted, for example, by operable association with a secretory signal sequence as is known in the art). Without being limited by any particular theory of the invention, according to this embodiment, administration to the skeletal muscle can result in secretion of the polypeptide into the systemic circulation and delivery to target tissue(s). Methods of delivering virus vectors to skeletal muscle are described in more detail herein.

The invention can also be practiced to produce antisense RNA, RNAi or other functional RNA (e.g., a ribozyme) for systemic delivery.

The invention also provides a method of treating and/or preventing congenital heart failure or PAD in a subject in need thereof, the method comprising administering a treatment or prevention effective amount of a virus vector of the invention to a mammalian subject, wherein the virus vector comprises a heterologous nucleic acid encoding, for example, a sarcoplasmic endoreticulum Ca²⁺-ATPase (SERCA2a), an angiogenic factor, phosphatase inhibitor I (I-1) and fragments thereof (e.g., I1C), RNAi against phospholamban; a phospholamban inhibitory or dominant-negative molecule such as phospholamban S16E, a zinc finger protein that regulates the phospholamban gene, β2-adrenergic receptor, β2-adrenergic receptor kinase (BARK), PI3 kinase, calsarcan, a β-adrenergic receptor kinase inhibitor (βARKct), inhibitor 1 of protein phosphatase 1 and fragments thereof (e.g., I1C), S100A1, parvalbumin, adenylyl cyclase type 6, a molecule that effects G-protein coupled receptor kinase type 2 knockdown such as a truncated constitutively active bARKct, Pim-1, PGC-1α, SOD-1, SOD-2, EC-SOD, kallikrein, HIF, thymosin-β4, mir-1, mir-133, mir-206, mir-208 and/or mir-26a.

In some embodiments, the invention further encompasses a method of treating and/or preventing a congenital neurodegenerative disorder (e.g., monogenic neurodegenerative disorder) in a subject in need thereof, the method comprising: administering a treatment or prevention effective amount of a virus vector of the invention to neural tissue (e.g., neuronal cells) of a subject, wherein the virus vector comprises a heterologous nucleic acid encoding a polypeptide, wherein the congenital neurodegenerative disorder is a result of a deficiency and/or defect in the polypeptide. Illustrative congenital neurodegenerative disorders and heterologous nucleic acids encoding polypeptides are described herein. Optionally, the polypeptide is secreted (e.g., a polypeptide that is a secreted polypeptide in its native state or that has been engineered to be secreted, for example, by operable association with a secretory signal sequence as is known in the art). In some embodiments, the subject is a human. In some embodiments, the subject is in utero. In some embodiments, the subject has or is at risk for a congenital (e.g., monogenic) neurodegenerative disorder. In some embodiments, the subject has or is at risk for mucopolysacharidosis or leukodystrophy.

Injectables can be prepared in conventional forms, either as liquid solutions or suspensions, solid forms suitable for solution or suspension in liquid prior to injection, or as emulsions. Alternatively, one may administer the virus vector and/or virus capsids of the invention in a local rather than systemic manner, for example, in a depot or sustained-release formulation. Further, the virus vector and/or virus capsid can be delivered adhered to a surgically implantable matrix (e.g., as described in U.S. Patent Publication No. 20040013645).

The virus vectors and/or virus capsids disclosed herein can be administered to the lungs of a subject by any suitable means, optionally by administering an aerosol suspension of respirable particles comprised of the virus vectors and/or virus capsids, which the subject inhales. The respirable particles can be liquid or solid. Aerosols of liquid particles comprising the virus vectors and/or virus capsids may be produced by any suitable means, such as with a pressure-driven aerosol nebulizer or an ultrasonic nebulizer, as is known to those of skill in the art. See e.g., U.S. Pat. No. 4,501,729. Aerosols of solid particles comprising the virus vectors and/or capsids may likewise be produced with any solid particulate medicament aerosol generator, by techniques known in the pharmaceutical art.

The virus vectors and virus capsids can be administered to tissues of the central nervous system (CNS) (e.g., brain, eye) and may advantageously result in broader distribution of the virus vector or capsid than would be observed in the absence of the present invention.

In particular embodiments, the delivery vectors of the invention may be administered to treat diseases of the CNS, including genetic disorders, neurodegenerative disorders, psychiatric disorders and tumors. Illustrative diseases of the CNS include, but are not limited to Alzheimer's disease, Parkinson's disease, Huntington's disease, Canavan disease, Leigh's disease, Refsum disease, Tourette syndrome, primary lateral sclerosis, amyotrophic lateral sclerosis, progressive muscular atrophy, Pick's disease, muscular dystrophy, multiple sclerosis, myasthenia gravis, Binswanger's disease, trauma due to spinal cord or head injury, Tay Sachs disease, Lesch-Nyan disease, epilepsy, cerebral infarcts, psychiatric disorders including mood disorders (e.g., depression, bipolar affective disorder, persistent affective disorder, secondary mood disorder), schizophrenia, drug dependency (e.g., alcoholism and other substance dependencies), neuroses (e.g., anxiety, obsessional disorder, somatoform disorder, dissociative disorder, grief, post-partum depression), psychosis (e.g., hallucinations and delusions), dementia, paranoia, attention deficit disorder, psychosexual disorders, sleeping disorders, pain disorders, eating or weight disorders (e.g., obesity, cachexia, anorexia nervosa, and bulimia) cancers and tumors (e.g., pituitary tumors) of the CNS, and congenital neurodegenerative disorders such as mucopolysacharidosis (including, but not limited to, Mucopolysaccharidosis Type I (also known as Hurler syndrome, Hurler-Scheie Syndrome, or Scheie syndrome, IDUA, alpha-L-iduronidase), Mucopolysaccharidosis Type II (also known as Hunter syndrome, IDS, I2L enzyme), Mucopolysaccharidosis Type III (also known as Sanfilippo syndrome, GNS [N-acetylglucosamine-6-sulfatase], HGSNAT [heparan-alpha-glucosaminide N-acetyltransferase], NAGLU [alpha-N-acetylglucosaminidase], and/or SGSH [sulfamidase]), Mucopolysaccharidosis Type IV (also known as Morquio syndrome, GALNS [galatosamine (N-acetyl)-6-sulfatase] and/or GLB1 [beta-galactosidase]), Mucopolysaccharidosis Type V (also known as Scheie syndrome, now a subgroup of type I, also IDUA, alpha-L-iduronidase), Mucopolysaccharidosis Type VI (also known as Maroteaux-Lamy syndrome, ARSB, arylsulfatase B), Mucopolysaccharidosis Type VII (also known as Sly syndrome, GUSB, beta-glucuronidase), Mucopolysaccharidosis Type IX (also known as Natowicz syndrome, HYAL1, hyaluronidase) and/or leukodystrophy (including, but not limited to, adult-onset autosomal dominant leukodystrophy (ADLD; LMNB1, lamin B1), Aicardi-Goutieres syndrome (TREX1, RNASEHSB, RNASEH2C, and/or RNASEH2A), Alexander disease (FRAP, glial fibrillary acidic protein), CADASIL (Notch3), Canavan disease (ASPA, aspartoacylase), CARASIL (HTRA1, serine protease HTRA1), cerebrotendinous xanthomatosis (“CTX,” CYP27A1, sterol 27-hydroxylase) childhood ataxia and cerebral hypomyelination (CACH)/vanishing white matter disease (VWMD) (eIF2B, eukaryotic initiation factor 2B), Fabry disease (GLA, alpha-galactosidase A), fucosidosis (FUCA1, alpha-L-fucosidase), GM1 gangliosidosis (GLB1, beta-galactosidase), L-2-hydroxyglutaric aciduria (L2HDGH, L-2-hydroxyglutarate dehydrogenase), Krabbe disease (GALC, galactocerebrosidase), megalencephalic leukoencephalopathy with subcortical cysts (“MLC,” MLC1 and/or HEPACAM), metachromatic leukodystrophy (ASA, arylsulphatase A), multiple sulfatase deficiency (“MSD,” SUMF1, sulfatase modifying factor 1 affecting all sulfatase enzymes), Pelizaeus-Merzbacher disease (also known as “X-linked spastic paraplegia,” PLP1 [X-linked proteolipid protein 1] and/or GJA12 [gap junction protein 12]), Pol III-Related Leukodystrophies (POLR3A and/or POLR3B), Refsum disease (PHYH, [phytanoyl-CoA hydroxylase] and/or Pex7 [PHYH importer into peroxisomes]), salla disease (also known as “free sialic acid storage disease,” SLC17A5, sialic acid transporter), Sjogren-Larsson syndrome (ÁLDH3A2, aldehyde dehydrogenase), X-linked adrenoleukodystrophy (“ALD,” ABCD1, peroxisomal ATPase Binding Cassette protein), Zellweger syndrome spectrum disorders (also known as peroxisomal biogenesis disorders, PEX1, PEX2, PEX3, PEX4, PEX5, PEX10, PEX11B, PEX12, PEX13, PEX14, PEX16, PEX19, PEX26), and the like.

Disorders of the CNS include ophthalmic disorders involving the retina, posterior tract, and optic nerve (e.g., retinitis pigmentosa, diabetic retinopathy and other retinal degenerative diseases, uveitis, age-related macular degeneration, glaucoma).

Most, if not all, ophthalmic diseases and disorders are associated with one or more of three types of indications: (1) angiogenesis, (2) inflammation, and (3) degeneration. The delivery vectors of the present invention can be employed to deliver anti-angiogenic factors; anti-inflammatory factors; factors that retard cell degeneration, promote cell sparing, or promote cell growth and combinations of the foregoing.

Diabetic retinopathy, for example, is characterized by angiogenesis. Diabetic retinopathy can be treated by delivering one or more anti-angiogenic factors either intraocularly (e.g., in the vitreous) or periocularly (e.g., in the sub-Tenon's region). One or more neurotrophic factors may also be co-delivered, either intraocularly (e.g., intravitreally) or periocularly.

Uveitis involves inflammation. One or more anti-inflammatory factors can be administered by intraocular (e.g., vitreous or anterior chamber) administration of a delivery vector of the invention.

Retinitis pigmentosa, by comparison, is characterized by retinal degeneration. In representative embodiments, retinitis pigmentosa can be treated by intraocular (e.g., vitreal administration) of a delivery vector encoding one or more neurotrophic factors.

Age-related macular degeneration involves both angiogenesis and retinal degeneration. This disorder can be treated by administering the inventive delivery vectors encoding one or more neurotrophic factors intraocularly (e.g., vitreous) and/or one or more anti-angiogenic factors intraocularly or periocularly (e.g., in the sub-Tenon's region).

Glaucoma is characterized by increased ocular pressure and loss of retinal ganglion cells. Treatments for glaucoma include administration of one or more neuroprotective agents that protect cells from excitotoxic damage using the inventive delivery vectors. Such agents include N-methyl-D-aspartate (NMDA) antagonists, cytokines, and neurotrophic factors, delivered intraocularly, optionally intravitreally.

In other embodiments, the present invention may be used to treat seizures, e.g., to reduce the onset, incidence and/or severity of seizures. The efficacy of a therapeutic treatment for seizures can be assessed by behavioral (e.g., shaking, ticks of the eye or mouth) and/or electrographic means (most seizures have signature electrographic abnormalities). Thus, the invention can also be used to treat epilepsy, which is marked by multiple seizures over time.

In one representative embodiment, somatostatin (or an active fragment thereof) is administered to the brain using a delivery vector of the invention to treat a pituitary tumor. According to this embodiment, the delivery vector encoding somatostatin (or an active fragment thereof) is administered by microinfusion into the pituitary. Likewise, such treatment can be used to treat acromegaly (abnormal growth hormone secretion from the pituitary). The nucleic acid sequences (e.g., GenBank Accession No. J00306) and amino acid sequences (e.g., GenBank Accession No. P01166; contains processed active peptides somatostatin-28 and somatostatin-14) of somatostatins are known in the art.

In particular embodiments, the vector can comprise a secretory signal as described, e.g., in U.S. Pat. No. 7,071,172.

In representative embodiments of the invention, the virus vector and/or virus capsid is administered to the CNS (e.g., to the brain or to the eye). The virus vector and/or capsid may be introduced into the spinal cord, brainstem (medulla oblongata, pons), midbrain (hypothalamus, thalamus, epithalamus, pituitary gland, substantia nigra, pineal gland), cerebellum, telencephalon (corpus striatum, cerebrum including the occipital, temporal, parietal and frontal lobes. cortex, basal ganglia, hippocampus and portaamygdala), limbic system, neocortex, corpus striatum, cerebrum, and/or inferior colliculus. The virus vector and/or capsid may also be administered to different regions of the eye such as the retina, cornea and/or optic nerve.

The virus vector and/or capsid may be delivered into the cerebrospinal fluid (e.g., by lumbar puncture) for more disperse administration of the delivery vector. The virus vector and/or capsid may further be administered intravascularly to the CNS in situations in which the blood-brain barrier has been perturbed (e.g., brain tumor or cerebral infarct).

The virus vector and/or capsid can be administered to the desired region(s) of the CNS by any route known in the art, including but not limited to, intrathecal, intracerebral, intraventricular, intravenous (e.g., in the presence of a sugar such as mannitol), intranasal, intra-aural, intra-ocular (e.g., intra-vitreous, sub-retinal, anterior chamber) and peri-ocular (e.g., sub-Tenon's region) delivery as well as intramuscular delivery with retrograde delivery to motor neurons.

In some embodiments, the virus vector or composition of the present invention may be delivered via an enteral, parenteral, intrathecal, intracisternal, intracerebral, intraventricular, intranasal, intra-aural, intra-ocular, peri-ocular, intrarectal, intramuscular, intraperitoneal, intravenous, oral, sublingual, subcutaneous and/or transdermal route. In some embodiments, the virus vector or composition of the present invention may be delivered intracranially and/or intraspinally.

In particular embodiments, the virus vector and/or capsid is administered in a liquid formulation by direct injection (e.g., stereotactic injection) to the desired region or compartment in the CNS. In other embodiments, the virus vector and/or capsid may be provided by topical application to the desired region or by intra-nasal administration of an aerosol formulation. Administration to the eye may be by topical application of liquid droplets. As a further alternative, the virus vector and/or capsid may be administered as a solid, slow-release formulation (see, e.g., U.S. Pat. No. 7,201,898).

In yet additional embodiments, the virus vector can used for retrograde transport to treat and/or prevent diseases and disorders involving motor neurons (e.g., amyotrophic lateral sclerosis (ALS); spinal muscular atrophy (SMA), etc.). For example, the virus vector can be delivered to muscle tissue from which it can migrate into neurons.

Having described the present invention, the same will be explained in greater detail in the following examples, which are included herein for illustration purposes only and are not intended to be limiting to the invention.

### EXAMPLES

### Example 1

The wild-type viral genome of the canonical adeno-associated virus (AAV) serotype 2 is a single-stranded (ss) DNA genome of approximately 4,700 nucleotides and contains multiple genes with overlapping reading frames. The ends of the genome are flanked by 145 nucleotide inverted terminal repeat (ITR) sequences that are predicted to fold back upon themselves to form hairpin structures (FIGS. 1A-1B). The Cap gene produces the capsid VP proteins and also contains the reading frame for AAP which helps in assembly of the capsid. The AAV2 Rep gene produces four proteins named for their approximate weights: Rep78, Rep68, Rep50, and Rep42. The small Reps, 50 and 42, can act as motor proteins to package nascent genomes into preformed capsids. The large Reps, 78 and 68, have endonuclease and ATP-dependent helicase functions that are used for genomic replication. These large Reps can initiate genome replication by binding to the Rep Binding Element (RBE) in the A region of the ITR. This initial binding may help to unwind the DNA strands and form a nicking stem that is cleaved by Rep at the dinucleotide TT terminal resolution site (trs). In addition, the large Rep proteins also make contact with the RBE′ region at the tip of the C-loop (FIGS. 1A-1B). The ITR plays a fundamental role in the life cycle of AAV by containing the replication of origin, packaging signals, and the ability to confer persistence to AAV genomes after infection.

For AAV serotypes 1-4 and 6-7, the predicted structure of the ITR is similar but there are sequence differences throughout, notably in the number of GAGC repeats in the RBE, the TTT or TCT at the RBE′, the nucleotides in the hairpin loops, and the nucleotides in the D-region that do not participate in the formation of the nicking stem (FIGS. 1A-1C). Even with these differences, the AAV2 Reps are capable of replicating and cross-packaging genomes from serotypes 1, 3, 4, and 6 into numerous, non-AAV2 capsids.

Currently, recombinant AAV (rAAV) vector production platforms rely on an AAV2 Rep-AAV2 ITR replication and packaging system. In rAAV, the internal genes of AAV are removed, leaving only the ITRs to flank the therapeutic cassette. Thus, in a clinical setting, patients receiving gene therapy are exposed not only to the capsid proteins, but also the native viral AAV2 ITR sequences. The impact of these sequences in cells has been historically understudied, but it is known that the AAV ITR may interact with a number of host proteins and may stimulate anti-viral and DNA damage response pathways (Julien et al. 2018 Sci. Rep. 8:210; Qing et al. 2001 J. Virol. 75:8968-76; Satkunanathan et al. 2017 Virology 510:46-54; Raj et al. 2001 Nature 412:914-7; Hirsch et al. 2011 PLoS One 6).

In this study, various cell lines were infected with AAV vectors containing the ITR sequences from AAV serotypes 1, 2, 3, 4, 6, and 7 and their ability to promote luciferase expression was measured in vitro. Additionally, the transcription start sites (TSS) for ITRs 1-4, 6 or 7 were determined using amplification of luciferase specific cDNA. Finally, floxed-luciferase mice were injected with ITR-cre recombinase vectors to assess ITR promoter ability in a mouse model to determine if non-ITR2 sequences could also elicit transgene expression in vivo.

Plasmid construction: The ITR sequences from AAV serotypes 1˜4 & 6-7 sequences were ordered from Genscript with unique restriction enzyme sites flanking the sequences for downstream cloning. These ITRs were ordered with one ITR per plasmid to prevent potential intermolecular recombination during synthesis and propagation. These plasmids were electroporated into SURE Electroporation-Competent Cells (Agilent, 200227). Colony plasmids were screened for intact ITR sequences using restriction enzymes specific to the ITR genotype and then cloned into a pUC19 backbone with a 20 nucleotide stuffer sequence chosen randomly from lambda phage DNA, followed by the reading frame for luciferase or cre recombinase, an SV40 early polyA signal, and 2172 nucleotides of lambda phage DNA stuffer sequence to bring the total length of the AAV vector genome to 4395 or 3778 bases, respectively. After plasmid construction, the ITR sequences were verified using the illustra TempliPhi Sequence Resolver Kit (GE Life Sciences, 28903529) followed by Sanger sequencing. After sequence confirmation, every subsequent plasmid prep was digested with multiple ITR-specific restriction enzymes to ensure the presence and stability of the ITR sequence.

Cell lines and virus product: HEK293, HeLa, and Huh7 cells were maintained at 37° C. in 5% CO2 in Dulbecco's modified Eagle's medium with 10% bovine calf serum and 1% penicillin-streptomycin. Recombinant AAV vectors were produced using the triple transfection method. Briefly, 15 cm plates of HEK293 cells at ˜80% confluency were transfected with ITR-containing luciferase or cre recombinase vector plasmids, an AAV helper plasmid containing AAV2 Rep and AAV1, 2, or 9 Cap genes, and the Ad helper plasmid pXX6-80. 2 days post-transfection, the cells were collected, lysed, and subjected to a CsCl gradient ultracentrifugation. Fractions corresponding to the highest concentration of virus were taken and dialyzed in PBS (Slide-A-Lyzer Dialyses Cassettes MWCO 30,000, Thermo Fisher, #66003). Virus titer was determined in triplicate by quantitative polymerase chain reaction (qPCR) using transgene or stuffer-sequence specific primers and a viral standard containing the same transgene or stuffer sequence. A new batch of virus was made for every triplicate experiment and viruses were only used in the same experiment if they had been produced and titered together, 5 batches of virus were made three times to test the AAV1-4 & 6 ITRs in triplicate for a total of 15 batches of virus.

In vitro infection and luciferase assays: For comparison of AAV2/2-ITR-luciferase and AAV2/2-CBA-luciferase, 3.2E5 HEK293 cells were plated per well in 12 well plates and were infected the following day with 1E5 vg/cell. 2 days post-infection, cells were lysed in 200 ul of Passive Lysis Buffer (PLB) for 20 min at room temperature. Cell lysate from AAV2/2-CBA-luciferase was diluted 1:50 in PLB. 25 ul of cell lysate was combined with 100 ul of luciferin (Luciferase Assay System, Promega, E1500) in a 96-well opaque white assay plate and luminescence was measured with the Perkin Elmer Victor³ plate reader. Relative Light Units (RLU) values from AAV2/2-CBA-luciferase were multiplied by their dilution factor. The nomenclature used here to denote the ITR and capsid serotype is AAVN/N is AAV(ITR genotype)/capsid serotype. For comparison of ITR-luciferase vectors, HEK293, HeLa, and Huh7 cells were plated individually into 6-well plates. The following day, the cells were infected with 2E5 vg/cell of AAV(N)/2-ITR-luciferase where N is the indicated ITR genotype. Two days later, the media was removed and the cells were lysed in 350 ul of Passive Lysis Buffer for 20 min at room temperature. The lysate was transferred to 1.5 ml tubes and spun at 13,000g for 10 min at 4° C. 25 ul of lysate was used in a BCA (Pierce™ BCA Protein Assay Kit, 23225) assay to determine total cellular protein concentration. 100 ul of cell lysate was combined with 100 ul of luciferin (Luciferase Assay System, Promega, E1500) in a 96 well opaque white assay plate and luminescence was measured with the Perkin Elmer Victor³ plate reader. Relative Light Units were normalized to total protein added.

Identification of transcription start sites: HEK293 cells were infected with AAV(1-4, 6 or 7)/2-ITR-luciferase at 2E5 vg/cell and cultured for three days prior to RNA harvest using a Qiagen RNeasy kit. Following the manufacturer's instructions from the 5′/3′ RACE 2nd generation kit (Roche, 3353621001), approximately 750 ng of RNA was reverse transcribed using a primer located within the luciferase coding sequence (5′-GTGACGAACGTGTACATCGAC-3′; SEQ ID NO:8). The synthesized cDNA was then purified using the QIAquick PCR purification kit (QIAGEN, 28104), and a polyA tail was added by terminal transferase per manufacturer's instructions. PCR was then conducted using the supplied forward primer, 5′-GACCACGCGTATCGATGTCGACTTTTTTTTTTTTTTTTV-3′ (SEQ ID NO:9), and a nested reverse primer in the luciferase coding sequence, 5′-CTTAGAACCGGTCGAACACCACGGTAGGCT-3′ (SEQ ID NO:10). The resulting PCR product was purified and used as a template for an additional PCR reaction with the kit supplied forward primer 5′-GACCACGCGTATCGATGTCGAC-3′ (SEQ ID NO:11), and another nested reverse primer within luciferase sequence 5′-TTAGTTGGATCCGGTTCCATCTTCCAGCGG-3′ (SEQ ID NO:12). The product was purified, normalized to 20 ng/ul EB buffer, and 25 ul were sent for EZ amplicon sequencing using next generation sequencing (NGS) by Genewiz. Resulting NGS data was analyzed by the UNC Lineberger Bioinformatics Core using STAR v2.7.0a (Dobin et al., 2013) to align reads to the reference genomes. The bam files were processed in R to tabulate the frequency of alignment start site. Sequences with multiple mismatches (>3) in the first 10 bases of alignment were filtered as we could not infer whether the alignment should start before or after the mismatches. Read pairs with insert size greater than expected (1000 bp) were also removed.

Animal Study: Animal experiments performed in this study were conducted with FVB.12956(B6)-Gt(ROSA)26Sor^{tm1(Luc)Kael}/J mice²² (Jackson Laboratories Stock No: 005125). The mice were maintained in accordance to National Institutes of Health guidelines, as approved by the UNC Institutional Animal Care and Use Committee (IACUC, Protocol number 19.023-1). Male mice were housed individually due to fighting. Each mouse was injected via tail vein with 100 ul of 1E9 viral genomes. Luciferase expression was imaged using the IVIS Kinetic (Caliper Lifesciences, Waltham, Mass.) following a 100 ul i.p. injection of D-luciferin substrate (XenoLight D-Luciferin, 122799, Perkin Elmer, Waltham, Mass.). Bioluminescent images were analyzed using Living Image (PerkinElmer, Waltham, Mass.). Acquisition was performed using Living Image software version 2.20 using photon values.

Statistics: All statistical calculations were performed using statistical software (GraphPad Prism 8.2). Data are presented as individual points with the group mean. Data for single comparisons were evaluated using an unpaired two tailed t test. Differences between different groups were considered to be statistically significant when p values were less than 0.05.

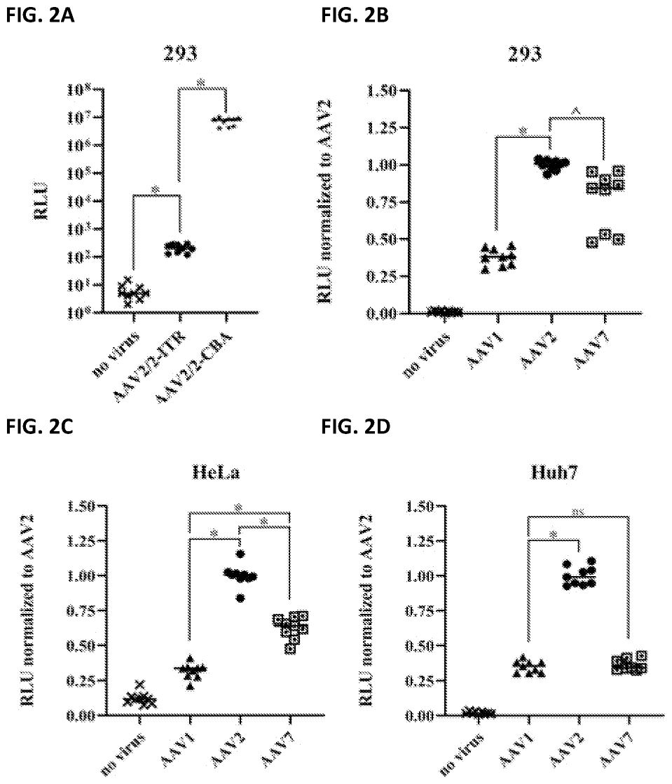

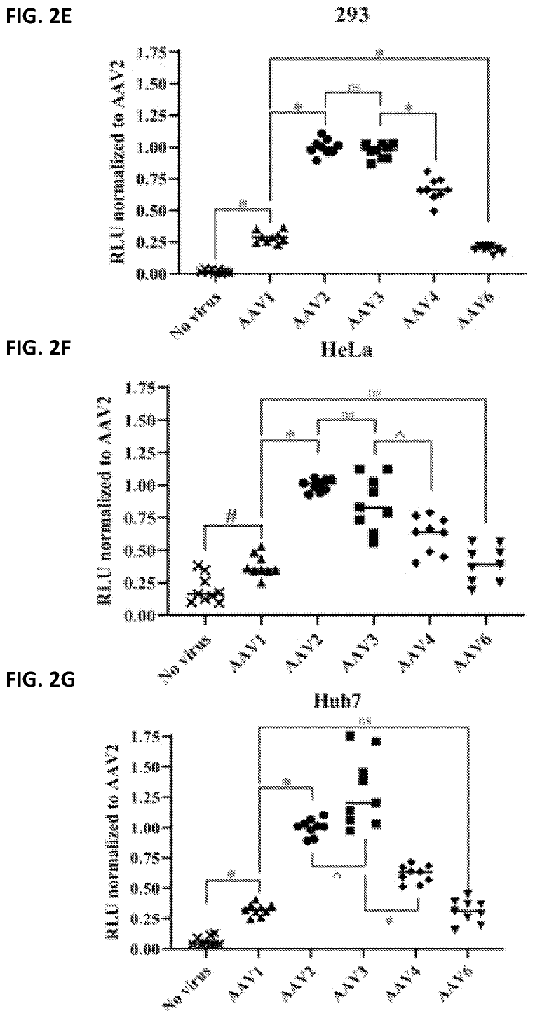

ITR serotype sequences have variable ability to promote luciferase expression in vitro. To determine the promoter activity of ITRs from various AAV genotypes, luciferase reporter vector plasmids were constructed using an AAV ITR sequence as the promoter. In vector plasmids, ITR sequences were assessed with multiple restriction enzymes to confirm the presence of the ITRs and their genotype identity. Initially, the activity of ITR2 was compared to the ‘strong’ CBA promoter. AAV2-ITR-luciferase and AAV2-CBA-luciferase were packaged into AAV2 capsids and used to infect HEK293 cells at 1E5 vg/cell. Two days post-infection, luciferase activity was measured and found to be over 4 logs higher from the CBA-promoted luciferase compared to ITR2-promoted luciferase (FIG. 2A). This demonstrated that ITR2 promoter activity could be successfully measured using a luciferase reporter system.

The use of ITR7 in an AAV2 Rep packaging system has yet to be reported in the literature. To test the feasibility of using ITR7 in combination with AAV2 Rep and an AAV2 capsid, ITR7 containing vectors were transfected into HEK293 cells with an adenoviral helper and pXR2. Resulting virus was titered by qPCR. ITR7 vectors had similar titers to ITR1 and ITR2 vectors made at the same time (Table 2). HEK293, HeLa, and Huh7 cells were infected with AAV1/2, AAV2/2, and AAV7/2-ITR-luciferase at 2E5 vg/cell. Luciferase activity was measured two days post-infection. Relative light units (RLU) were normalized to total amount of cellular protein added to the luciferase assay as determined by a BCA assay and then further normalized to ITR2 RLU values (FIGS. 2B-2D). RLUs from ITR1-promoted luciferase were consistently lower than that of ITR2 across all three cells lines (p<0.0001). In HEK293 cells, ITR1 had an average of 29% activity compared to ITR2 (FIG. 2B). This activity was slightly higher in HeLa cells at 35% (FIG. 2C) and in Huh7 cells at 32% (FIG. 2D). In contrast, ITR7 displayed different promoter activity across the cell lines. In Huh7 cells, ITR7 and ITR1 had the same expression level, 33% and 31% respectively, compared to ITR2 (FIG. 2D), but ITR7 had higher expression than ITR1 in HeLa cells at 62% (p<0.0001) (FIG. 2C). In HEK293 cells, ITR7-promoted luciferase activity rose to an average of 83%, with one preparation of virus having reduced activity compared to the other two preparations of virus (FIG. 2B), but this over all activity was still lower than that of ITR2 (p=0.0029).

To determine if ITRs 3, 4, and 6 also displayed promoter activity in these cell lines, ITR-luciferase plasmids were used to create AAV(1-4, 6)/2-ITR-luciferase vectors where AAVN/N is AAV(ITR genotype)/capsid serotype. All ITRs were able to be replicated and packaged by AAV2 Rep and batch titers were within 4-fold of each other. Of note, under these replication, packaging and purification conditions, titers from ITR2 containing vector plasmids were usually the highest, while ITR6 or ITR7 were always the lowest (Table 2).

HEK293, HeLa, and Huh7 cells were infected with AAV(1-4, 6 or 7)/2-ITR-luciferase vectors at 2E5 vg/cell. Luciferase activity was measured as described above. ITRs 3, 4, and 6 also resulted in luciferase activity in the cell lines tested, but to varying degrees (FIGS. 2E-2G). In all three cells lines, ITR2 and ITR3 resulted in the highest luciferase activity. Only in Huh7 cells was there a significant difference between ITR2 and ITR3 (p=0.0082), with ITR3 averaging 30% more activity than ITR2 (FIG. 2G). Across all cell types, ITR1 and ITR6 had the lowest activity and were not statistically different than each other, except in HEK293 cells in which ITR1 was 10% lower than ITR6 (p<0.0001) with a mean of 19% compared to 29% (FIG. 2E). ITR4 consistently had 62-66% luciferase activity compared to ITR2 (FIG. 2E-2G) and was significantly lower than ITR3 as well in all three cell lines (p<0.01). Thus, the observed activity from the ITRs fell into three classes: Class I ITRs with the highest relative activity: ITR2 and ITR3, Class II with an intermediate level of activity: ITR4, and Class III which had the lowest activity: ITR1 and ITR6. Of all the ITRs tested, only ITR7 showed cell-specific activity (FIGS. 2B-2D).

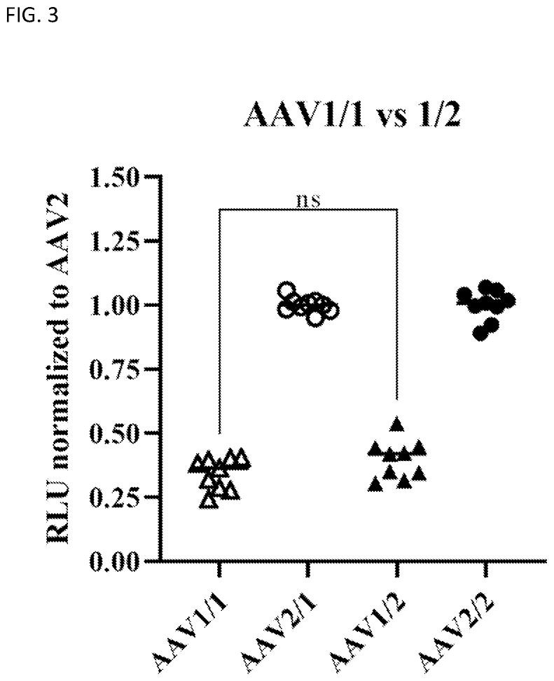

The differing levels of luciferase activity from ITR sequences 1-4, 6 implied that the ITR sequence itself was a significant determinate of luciferase activity, but alternatively, the high luciferase activity from the ITR2-containing vectors could be due to a capsid-specific interaction since this ITR was paired with its cognate capsid. To test if ITR sequences packaged into their corresponding capsid influenced luciferase production, ITR1 and ITR2 luciferase vectors were packaged into AAV1 capsids and used to infect HEK293 cells at 2E5 vg/cell. While the overall activity was reduced compared to AAV1/2 and AAV2/2, luciferase activity from AAV1/1-ITR-luciferase vectors was still lower than AAV2/1 vectors. When normalized to AAV2, the activity was equivalent, regardless of the capsid used (FIG. 3). Hence, ITR1 is still a Class III ITR, even when paired with its cognate capsid.

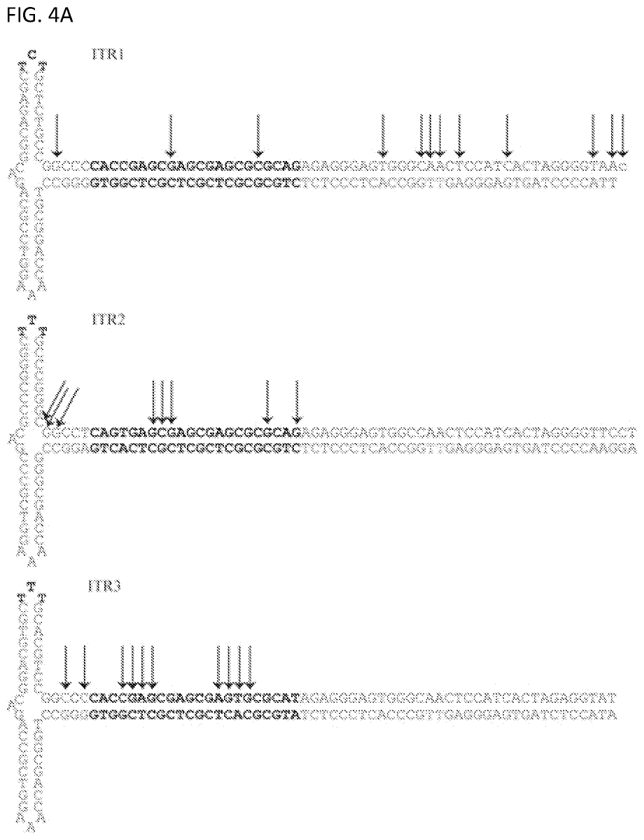

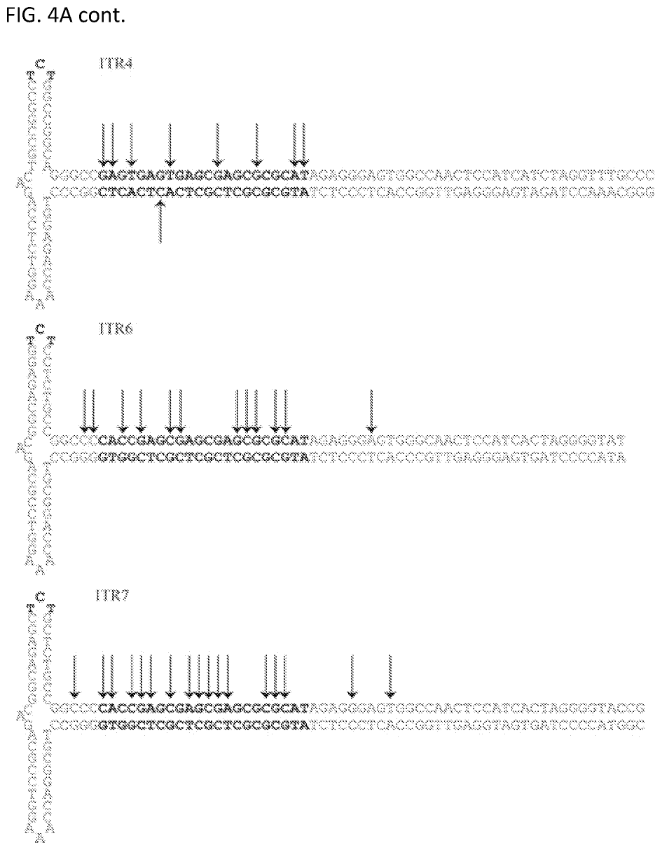

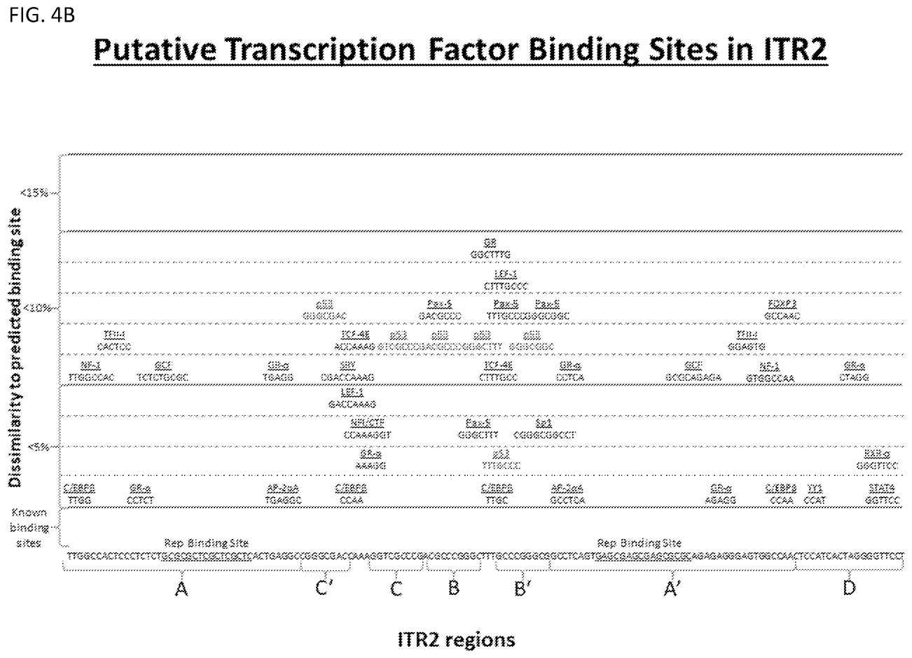

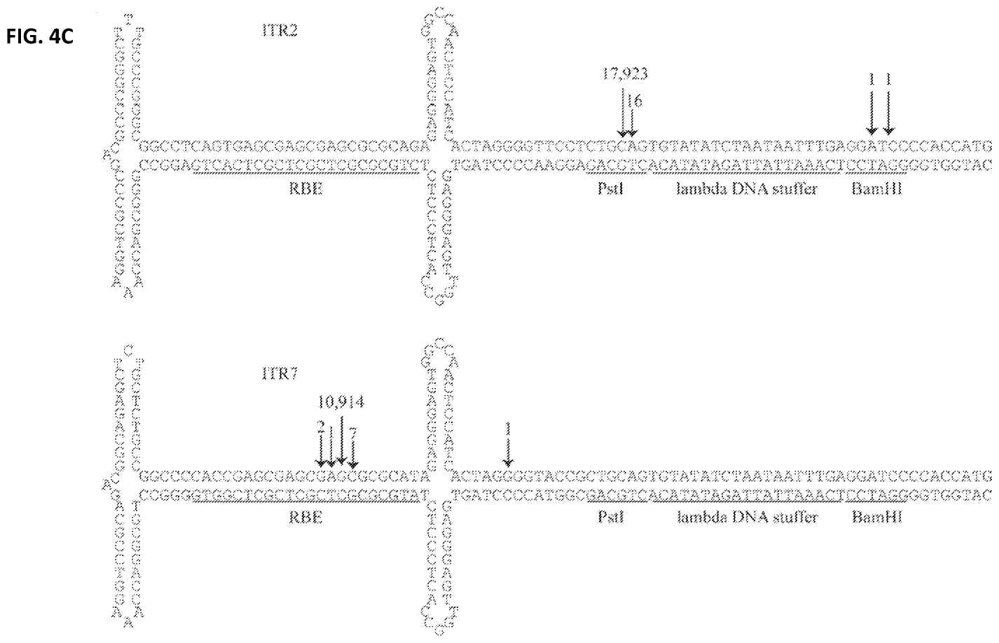

The ITR sequences contain multiple transcription start sites (TSS). The ITR sequences of all the genotypes tested are high in CG content (64-70%) and lack a traditional TATA-box consensuses sequence. To determine if the luciferase transcripts were originating from a single, focused TSS or multiple, dispersed TSSs, 5′ rapid amplification of cDNA ends (RACE) was employed to find the originating nucleotide position(s). HEK293 cells were infected with AAV(1-4, 6 or 7)/2-ITR-luciferase at 2E5 vg/cell. Three days post-infection, total RNA was isolated and reverse transcribed using a 5′ Rapid amplification of cDNA ends (RACE) kit. Luciferase-specific cDNA was amplified and analyzed by next-generation sequencing (NGS). For all ITRs, multiple TSSs were found within each sequence and tended to cluster at the RBE, although ITR1 had more widespread start sites than the other ITRs (FIG. 4A). For each ITR, 3-4 nucleotides represented that majority of reads, but these hot spots were different for each ITR. These results indicate that the transcripts for ITR-promoted luciferase can originate from multiple start sites within the ITR.

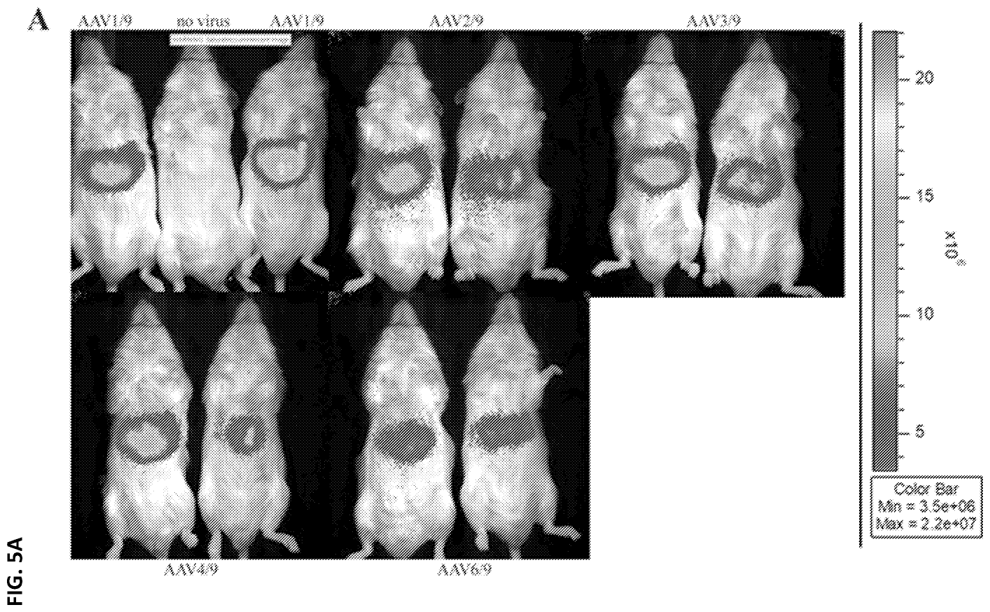

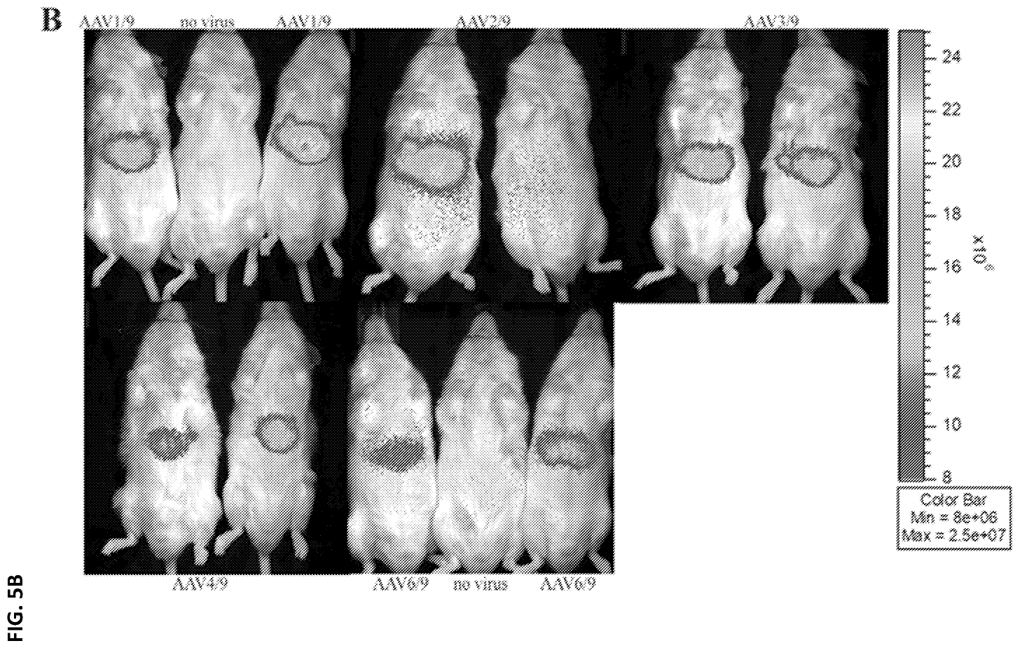

Cre-recombinase driven by ITRs sequences 1-6 is capable of activating luciferase production in vivo. To see how the in vitro findings translated to an in vivo model, the ITR promoter ability was tested in a floxed luciferase reporter mouse strain. The FVB.129S6(B6)-Gt(ROSA)26Sor^{tm1(Luc)Kael}/J mouse line contains a luciferase open reading frame inserted into the ROSA26 locus. Luciferase expression is prevented by a loxP-stop-LoxP sequence which can be removed by cre recombinase. 4-6 week old male mice were injected with 100 ul of 1E9 vg of AAV(1-4, 6)/9-ITR-cre recombinase via the tail vein (N=2). AAV9 was chosen for its ability to highly transduce most tissues, thus likely the best to identify tissue-specific differences, if any, between the ITR promoters. As a positive control, two mice were injected with AAV2/9-CMV-Cre vectors. Mice were imaged at 3, 5, 7, and 9 weeks post-injection. By three weeks post-injection, luciferase activity could be observed in the abdominal area of all mice (FIG. 5A). As expected, the positive control mice which had been injected with CMV promoted cre recombinase had more recombined cells expressing luciferase and under the same imaging setting these mice entirely saturated the camera. By four weeks post-injection, luciferase signal from one of the mice injected with AAV2/9-ITR-cre recombinase could no longer be detected. During the 9-week time course, luciferase signal remained steady in the remaining mice (FIG. 5B). The loss of expression from the mouse injected with AAV2/9-ITR-cre recombinase was likely due to capsid antigen reactive CD8+ T-cells, but this was not specifically investigated.

ITR serotype sequence influences promoter activity in vitro. The ITR promoter activity for ITRs 1-4 and 6 was consistent enough that they could be broken into three Classes: I, II, and III, with I being the highest activity. Across each cell line, ITR2 and ITR3 (Class I) consistently had the highest values for luciferase activity, implying these sequences also have more promoter activity than the other ITRs tested, while ITR1 and ITR6 (Class III) had the lowest (FIGS. 2A-2G). The similar expression values for ITR1 and ITR6 would be expected given the high degree of similarity between the two sequences, which differ from each other only by the last nucleotide in the D-region and only 4 nucleotides outside of the D region (FIG. 1C). A sequence analysis between Class I and Class III sequences revealed several points of variance that could explain the different activities (FIG. 1C). Specifically, ITR2 and ITR3 contained a TTT sequence in the RBE′, whereas ITR1 and ITR6 contain TCT. There was also a consistent difference in the B-loop (positions 45:59 and 46:60) and C-loop (68:80 and 70:78), and a C to G change in the tips of the nicking stem loops at positions 3 and 122. ITR7 is also similar to the ITR1 sequence, but had a different promoter activity profile (FIGS. 2B-2D). Since there are only a few nucleotides that differ between ITR1 and ITR7, a mutational analysis may be able to find the specific sequence(s) involved in the differential expression of ITR7-promoted luciferase in various cell types. The T:A pair in ITR7 at position 110:15 is the same pair seen at ITR3, 4, and 6 (FIG. 1C), so it is unlikely to be involved in the varying levels of luciferase activity we observed across the cell types tested. Like ITR2, 3 and 4, ITR7 also has a G near the nicking site at position 3 and a C at position 122, so these nucleotides may influence promoter strength. Another variable region of interest that could be influencing luciferase expression among the ITRs is the last 11 nucleotides of the D region where only a CTAG motif is conserved, but there is no readily discernable pattern between the different classes of ITRs. Still, this region could be of interest since several host proteins have been shown to interact with the D region of ITR2. The question of which sequences have effects on transgene production may be addressed with position specific ITR mutants, but given that complex DNA secondary structure may play diverse roles in transcription, this question may be difficult to unravel fully.

Under the conditions used here, ITR1 was still a Class III, even when packaged into an AAV1 capsid. This argues against an ITR-capsid interaction as having a strong influence on promoter activity. In this study, an AAV2 Rep was used, so it may still be that having the cognate Rep for these ITR sequences could influence various aspects of replication, packaging, and transducing units of rAAV.

Start sites for ITR-promoted luciferase transcripts. Previous work identified the A region of ITR2 as important for ITR2-promoted GFP transgene expression (Haberman et al. 2000 J. Virol. 74:8732-8739). Here it was also found that the A region was hotspot for transcriptional activity, but by using NGS it was possible to identify multiple starts and transcription factor binding sites throughout the ITR sequences, primarily focused within a 40 base-pair region that included the RBE (FIGS. 4A-4C). Lacking a traditional TATA-box within the defined ITR sequence, but enriched in cytosine and guanine, these sequences bear striking similarity to the transcriptionally active CpG islands (CGI) found in vertebrate genomes. It is now appreciated that CGIs are the most common promoter type in the vertebrate genome, occurring at 60-70% of annotated genes. CGIs are commonly defined as sequences with a C+G ratio of greater than 50% and an observed-to-expected CpG dinucleotides at 60% or higher. The AAV ITR sequences fit this definition both in C+G content and CpG frequency (Table 3). CGIs are often origins of replication and are generally associated with multiple TSSs dispersed over a 50-100 base-pair region. This is in contrast to promoters with a single, focused TSS that are more commonly associated with specifically positioned core promoter elements including the TATA-box, INR, TCT, and XCPE motifs. The data from this study supports a hypothesis that the ITR sequences from the AAV genotypes examined are functioning as CGI type promoters in the context of transgene promotion. That said, another interpretation of this data could be that these TSS are actually arising from distinct and variable episomal sequences. Since the resulting sequence that arises from the recombination of the two ITR ends after infection is variable, it may be that each episome has a different start site.

ITR1-4, 6 are capable of promoting cre recombinase in a mouse model. The in vivo data demonstrates that ITRs 1-4, 6 were able to promote high enough levels of cre recombinase to induce recombination and luciferase production. In this strain of mice, all ITR sequences 1-4, 6 were active promoters and this may have important implications for gene therapy applications. These mice were injected with 1E11 vg which an approximate equivalent to 5E12 vg/kg and thus a clinically relevant dose. In the context of a strong ubiquitous promoter, these ITR sequences would likely have no effect on overall transgene production, but there are scenarios in which more targeted or sensitive applications could be affected, such as when using AAV delivered cre recombinase or CRISPR.

More importantly, the bidirectional activity of the ITR2 promoter may be inducing the dsRNA response pathway. A prior study found that AAV transduction stimulated MDA5, a dsRNA response protein that recognizes dsRNA products over 2000 nucleotides long, at 8 days post-infection (Shao et al. 2018 JCI insight 3(12):e120474). It was proposed that the promoter activity of the ITR when in an episome confirmation may be driving minus strand RNA production which could bind to positive strand RNA and accumulate in transduced cells. In this scenario, a promoter with less activity would be desirable to help blunt this arm of the innate immune response. Eliminating all the CpG islands in the ITR would necessitate changing the RBE sequence, which has 5 CpGs, such that Rep could no longer efficiently bind it. Other strategies such as adding insulating sequences that flank the ITRs to prevent transcription read through may be fruitful.

The data presented here show that the ITRs sequences from AAV serotypes 1-4, 6 and 7 have inherent promoter activity and this promoter activity is not at equal strength amongst the ITRs. Specifically, ITR2 and ITR3 sequences resulted in higher luciferase expression across multiple cell types when compared to ITRs 1, 4, and 6. ITR7 was the only ITR to display cell-specific differences in luciferase expression. The TSS were mapped to multiple locations within each ITR sequence, of which the bulk originated from a 40 base-pair region that contained the RBE. In vivo, all the ITRs tested had the ability to promote cre recombinase at high enough levels to induce cre-mediated recombination by 3 weeks post-injection. These data may help inform vector design strategies when sensitive or cell-specific therapies are needed. Thus, this study shows for the first time that the ITR sequences for AAVs 1-4, 6, and 7 have varying ability to promote transgene expression in vitro and that these sequences contain multiple TSS. Additionally, a sensitive reporter mouse strain was utilized to demonstrate that at clinically relevant doses, the ITRs 1˜4 & 6 have the ability to promote enough cre recombinase protein in vivo to have biological effects at the cellular level.

The foregoing is illustrative of the present invention, and is not to be construed as limiting thereof.

TABLE 1
AAV Genomes

| AAV Serotypes/Isolates | GenBank Accession Number |
|---|---|
| Clonal Isolates | |
| Avian AAV | AY186198, AY629583, |
| ATCC VR-865 | NC_004828 |
| Avian AAV | NC_006263, AY629583 |
| strain DA-1 | |
| Bovine AAV | NC_005889, AY388617 |
| AAV4 | NC_001829 |
| AAV5 | AY18065, AF085716 |
| Rh34 | AY243001 |
| Rh33 | AY243002 |
| Rh32 | AY243003 |
| AAV10 | AY631965 |
| AAV11 | AY631966 |
| AAV12 | DQ813647 |
| AAV13 | EU285562 |
| Clade A | |
| AAV1 | NC_002077, AF063497 |
| AAV6 | NC_001862 |
| Hu.48 | AY530611 |
| Hu 43 | AY530606 |
| Hu 44 | AY530607 |
| Hu 46 | AY530609 |
| Clade B | |
| Hu19 | AY530584 |
| Hu20 | AY530586 |
| Hu23 | AY530589 |
| Hu22 | AY530588 |
| Hu24 | AY530590 |
| Hu21 | AY530587 |
| Hu27 | AY530592 |
| Hu28 | AY530593 |
| Hu29 | AY530594 |
| Hu63 | AY530624 |
| Hu64 | AY530625 |
| Hu13 | AY530578 |
| Hu56 | AY530618 |
| Hu57 | AY530619 |
| Hu49 | AY530612 |
| Hu58 | AY530620 |
| Hu34 | AY530598 |
| Hu35 | AY530599 |
| AAV2 | NC_001401 |
| Hu45 | AY530608 |
| Hu47 | AY530610 |
| Hu51 | AY530613 |
| Hu52 | AY530614 |
| Hu T41 | AY695378 |
| Hu S17 | AY695376 |
| Hu T88 | AY695375 |
| Hu T71 | AY695374 |
| Hu T70 | AY695373 |
| Hu T40 | AY695372 |
| Hu T32 | AY695371 |
| Hu T17 | AY695370 |
| Hu LG15 | AY695377 |
| Clade C | |
| AAV 3 | NC_001729 |
| AAV 3B | NC_001863 |
| Hu9 | AY530629 |
| Hu10 | AY530576 |
| Hu11 | AY530577 |
| Hu53 | AY530615 |
| Hu55 | AY530617 |
| Hu54 | AY530616 |
| Hu7 | AY530628 |
| Hu18 | AY530583 |
| Hu15 | AY530580 |
| Hu16 | AY530581 |
| Hu25 | AY530591 |
| Hu60 | AY530622 |
| Ch5 | AY243021 |
| Hu3 | AY530595 |
| Hu1 | AY530575 |
| Hu4 | AY530602 |
| Hu2 | AY530585 |
| Hu61 | AY530623 |
| Clade D | |
| Rh62 | AY530573 |
| Rh48 | AY530561 |
| Rh54 | AY530567 |
| Rh55 | AY530568 |
| Cy2 | AY243020 |
| AAV7 | AF513851 |
| Rh35 | AY243000 |
| Rh37 | AY242998 |
| Rh36 | AY242999 |
| Cy6 | AY243016 |
| Cy4 | AY243018 |
| Cy3 | AY243019 |
| Cy5 | AY243017 |
| Rh13 | AY243013 |
| Clade E | |
| Rh38 | AY530558 |
| Hu66 | AY530626 |
| Hu42 | AY530605 |
| Hu67 | AY530627 |
| Hu40 | AY530603 |
| Hu41 | AY530604 |
| Hu37 | AY530600 |
| Rh40 | AY530559 |
| Rh2 | AY243007 |
| Bb1 | AY243023 |
| Bb2 | AY243022 |
| Rh10 | AY243015 |
| Hu17 | AY530582 |
| Hu6 | AY530621 |
| Rh25 | AY530557 |
| Pi2 | AY530554 |
| Pi1 | AY530553 |
| Pi3 | AY530555 |
| Rh57 | AY530569 |
| Rh50 | AY530563 |
| Rh49 | AY530562 |
| Hu39 | AY530601 |
| Rh58 | AY530570 |
| Rh61 | AY530572 |
| Rh52 | AY530565 |
| Rh53 | AY530566 |
| Rh51 | AY530564 |
| Rh64 | AY530574 |
| Rh43 | AY530560 |
| AAV8 | AF513852 |
| Rh8 | AY242997 |
| Rh1 | AY530556 |
| Clade F | |
| AAV9 (Hu14) | AY530579 |
| Hu31 | AY530596 |
| Hu32 | AY530597 |

TABLE 2
Titers from vector preps using pXX6-80 and pXR2 helper
plasmids in vg/ul

| Batch 1 | | Batch 2 | | Batch 3 | |
|---|---|---|---|---|---|
| AAV1 | 8.88E+08 | AAV2 | 1.39E+09 | AAV2 | 2.92E+09 |
| AAV2 | 8.66E+08 | AAV1 | 1.11E+09 | AAV1 | 2.77E+09 |
| AAV7 | 7.28E+08 | AAV7 | 1.05E+09 | AAV7 | 1.87E+09 |
| AAV2 | 3.04E+08 | AAV2 | 8.72E+08 | AAV2 | 1.01E+09 |
| AAV4 | 2.42E+08 | AAV1 | 7.65E+08 | AAV4 | 9.23E+08 |
| AAV3 | 2.11E+08 | AAV4 | 6.89E+08 | AAV1 | 8.15E+08 |
| AAV1 | 1.97E+08 | AAV3 | 3.79E+08 | AAV3 | 5.83E+08 |
| AAV6 | 1.55E+08 | AAV6 | 2.23E+08 | AAV6 | 4.18E+08 |

TABLE 3
C + G content and Observed to Expected ratio of CpGs in
AAV 5′ ITR sequences

| AAV serotype | C + G content | observed-to-expected CpG ratio |
|---|---|---|
| AAV1 | 68.5% | 83.4% |
| AAV2 | 70.3% | 94.8% |
| AAV3 | 64.3% | 94.7% |
| AAV4 | 64.49% | 66.4% |
| AAV6 | 67.1% | 86.9% |
| AAV7 | 68.3% | 88.8% |

C+G content was calculated (C+G)/N where C is the number of cytosines, G is the number of guanines, and N is the number of nucleotides in the 5′ ITR sequence of the indicated AAV serotype. Observed-to-expected CpG ratio was calculated using the formula by Gardiner-Garden et al 1987: ((CpGs)/(C*G))*N where CpG is the number of observed CpGs, C is the number of cytosines, G is the number of guanines, and N is the number of nucleotides in the sequence.

SEQUENCES
SEO ID NO: 1. AAV2 ITR (ITR2); (NCBI Reference Sequence NC_001401.2)
TTGGCCACTCCCTCTCTGCGCGCTCGCTCGCTCACTGAGGCCGGGCGACCAAAGG
TCGCCCGACGCCCGGGCTTTGCCCGGGCGGCCTCAGTGAGCGAGCGAGCGCGCA
GAGAGGGAGTGGCCAACTCCATCACTAGGGGTTCCT
SEO ID NO: 2. AAV1 ITR (ITR1); NCBI Reference Sequence: NC_002077.1.
Also, GenBank: AF063497.1
TTGCCCACTCCCTCTCTGCGCGCTCGCTCGCTCGGTGGGGCCTGCGGACCAAAGG
TCCGCAGACGGCAGAGCTCTGCTCTGCCGGCCCCACCGAGCGAGCGAGCGCGCA
GAGAGGGAGTGGGCAACTCCATCACTAGGGGTAATC
SEQ ID NO: 3. AAV3B ITR (ITR3); GenBank: AF028705.1
TGGCCACTCCCTCTATGCGCACTCGCTCGCTCGGTGGGGCCTGGCGACCAAAGGT
CGCCAGACGGACGTGCTTTGCACGTCCGGCCCCACCGAGCGAGCGAGTGCGCAT
AGAGGGAGTGGCCAACTCCATCACTAGAGGTATGG
SEQ ID NO: 4. AAV4 ITR (ITR4); NCBI Reference Sequence: NC_001829.1.
Also, GenBank: U89790.1
TTGGCCACTCCCTCTATGCGCGCTCGCTCACTCACTCGGCCCTGGAGACCAAAGG
TCTCCAGACTGCCGGCCTCTGGCCGGCAGGGCCGAGTGAGTGAGCGAGCGCGCA
TAGAGGGAGTGGCCAACTCCATCATCTAGGTTTGCC
SEQ ID NO: 5; AAV6 ITR (ITR6); GenBank: AF028704 1
TTGGCCACTCCCTCTCTGCGCGCTCGCTCGCTCACTGAGGCCGGGCGACCAAAGG
TCGCCCGACGCCCGGGCTTTGCCCGGGCGGCCTCAGTGAGCGAGCGAGCGCGCA
GAGAGGGAGTGGCCAACTCCATCACTAGGGGTTCCT
SEQ ID NO: 6. AAV7 ITR (ITR7); GenBank: MP471038.1
TTGGCCACTCCCTCTATGCGCGCTCGCTCGCTCGGTGGGGCCTGCGGACCAAAGG
TCCGCAGACGGCAGAGCTCTGCTCTGCCGGCCCCACCGAGCGAGCGAGCGCGCA
TAGAGGGAGTGGCCAACTCCATCACTAGGGGTACC
SEQ ID NO: 7. Consensus ITR
TTGSCCACTCCCTCTMTGCGCRCTCGCTCRCTCRSTSGSGCCKGSVGACCAAAGGT
CBSCMGACKSMMGDGSTYTSCHCKKSMGGCSCSASYGAGYGAGCGAGYGCGCAK
AGAGGGAGTGGSCAACTCCATCAYYWRRGKTWN
SEQUENCE ALIGNMENT of SEQ ID NOs: 1-6

| | | |
|---|---|---|
| AAV4_ITR | TTGGCCACTCCCTCTATGCGCGCTCGCTCACTCACTCGGCCCTGGAGACCAAAGGTCTCC | 60 |
| AAV2_ITR | TTGGCCACTCCCTCTCTGCGCGCTCGCTCGCTCACTGAGGCCGGGCGACCAAAGGTCGCC | 60 |
| AAV6_ITR | TTGGCCACTCCCTCTCTGCGCGCTCGCTCGCTCACTGAGGCCGGGCGACCAAAGGTCGCC | 60 |
| AAV3b_ITR | -TGGCCACTCCCTCTATGCGCACTCGCTCGCTCGGTGGGGCCTGGCGACCAAAGGTCGCC | 60 |
| AAV1_ITR | TTGCCCACTCCCTCTCTGCGCGCTCGCTCGCTCGGTGGGGCCTGCGGACCAAAGGTCCGC | 60 |
| AAV7_ITR | TTGGCCACTCCCTCTATGCGCGCTCGCTCGCTCGGTGGGGCCTGCGGACCAAAGGTCCGC | 60 |
| | ** *********** ***** ******* *** * * ** * *********** * | |
| AAV4_ITR | AGACTGCCGGCCTCTGGCCGGCAGGGCCGAGTGAGTGAGCGAGCGCGCATAGAGGGAGTG | 120 |
| AAV2_ITR | CGACGCCCGGGCTTTGCCCGGGCGGCCTCAGTGAGCGAGCGAGCGCGCAGAGAGGGAGTG | 120 |
| AAV6_ITR | CGACGCCCGGGCTTTGCCCGGGCGGCCTCAGTGAGCGAGCGAGCGCGCAGAGAGGGAGTG | 120 |
| AAV3b_ITR | AGACGGACGTGCTTTGCACGTCCGGCCCCACCGAGCGAGCGAGTGCGCATAGAGGGAGTG | 119 |
| AAV1_ITR | AGACGGCAGAGCTCTGCTCTGCCGGCCCCACCGAGCGAGCGAGCGCGCAGAGAGGGAGTG | 120 |
| AAV7_ITR | AGACGGCAGAGCTCTGCTCTGCCGGCCCCACCGAGCGAGCGAGCGCGCATAGAGGGAGTG | 120 |
| | *** * ** ** * ** * * *** ******* ***** ******** | |
| AAV4_ITR | GCCAACTCCATCATCTAGGTTTGCC | 145 |
| AAV2_ITR | GCCAACTCCATCACTAGGGGTTCCT | 145 |
| AAV6_ITR | GCCAACTCCATCACTAGGGGTTCCT | 145 |
| AAV3b_ITR | GCCAACTCCATCACTAGAGGTATGG | 144 |
| AAV1_ITR | GGCAACTCCATCACTAGGGGTAATC | 145 |
| AAV7_ITR | GCCAACTCCATCACTAGGGGTACC- | 144 |
| | * *********** * * | |

1. A polynucleotide comprising at least one synthetic adeno-associated virus (AAV) inverted terminal repeat (ITR), wherein said ITR comprises:
(a) an AAV rep binding element (RBE);
(b) a B-loop;
(c) a C-loop;
(d) one or more nicking-stem loops;
(e) a D-region;
(f) an AAV terminal resolution sequence; and
(g) an AAV RBE′ element;
wherein (a)-(g) are from any AAV serotype that is not AAV2 or AAV3, and
(i) wherein the RBE′ element comprises a non-complementary loop TTT sequence at a position that corresponds to nucleotide positions 73 to 75, wherein the nucleotide numbering is based on the nucleotide sequence of SEQ ID NO:1;
(ii) wherein the B-loop comprises a nucleotide sequence that has 80% sequence identity to the nucleotide sequence of the B-loop of ITR2 or ITR3 at a position that corresponds to nucleotide positions 43-61, wherein the nucleotide numbering is based on the nucleotide sequence of SEQ ID NO:1;
(iii) wherein the C-loop comprises a nucleotide sequence that has 80% sequence identity to the nucleotide sequence of the C-loop of ITR2 or ITR3 at a position that corresponds to nucleotide positions 65-83, wherein the nucleotide numbering is based on the nucleotide sequence of SEQ ID NO:1;
(iv) wherein the D-region comprises a nucleotide sequence that has 80% sequence identity to the nucleotide sequence of the D-region of ITR2 or ITR3 at a position that corresponds to nucleotide positions 125-145, wherein the nucleotide numbering is based on the nucleotide sequence of SEQ ID NO:1; and/or
(v) wherein at least one of the one or more nicking-stem loops comprises a G substitution at a position that corresponds to nucleotide position 4 and/or a C substitution at a position that corresponds to nucleotide position 122, wherein the nucleotide numbering is based on the nucleotide sequence of SEQ ID NO:1.

2. The polynucleotide of claim 1, wherein said ITR has enhanced transcription function over wildtype AAV serotype ITR.

3. A polynucleotide comprising at least one synthetic adeno-associated virus (AAV) inverted terminal repeat (ITR), wherein said ITR comprises:
(a) an AAV rep binding element (RBE);
(b) a B-loop;
(c) a C-loop;
(d) one or more nicking-stem loops;
(e) a D-region;
(f) an AAV terminal resolution sequence; and
(g) an AAV RBE′ element;
wherein (a)-(g) are from any AAV serotype that is not AAV1 or AAV6, and
(i) wherein the RBE′ element comprises a non-complementary loop TCT sequence at a position that corresponds to nucleotide positions 73 to 75, wherein the nucleotide numbering is based on the nucleotide sequence of SEQ ID NO:1;
(ii) wherein the B-loop comprises a nucleotide sequence that has 80% sequence identity to the nucleotide sequence of the B-loop of ITR1 or ITR6 at a position that corresponds to nucleotide positions 43-61, wherein the nucleotide numbering is based on the nucleotide sequence of SEQ ID NO:1;
(iii) wherein the C-loop comprises a nucleotide sequence that has 80% sequence identity to the nucleotide sequence of the C-loop of ITR1 or ITR6 at a position that corresponds to nucleotide positions 65-83, wherein the nucleotide numbering is based on the nucleotide sequence of SEQ ID NO:1;
(iv) wherein the D-region comprises a nucleotide sequence that has 80% sequence identity to the nucleotide sequence of the D-region of ITR1 or ITR6 at a position that corresponds to nucleotide positions 125-145, wherein the nucleotide numbering is based on the nucleotide sequence of SEQ ID NO:1; and/or
(v) wherein at least one of the one or more nicking-stem loops comprises a C substitution at a position that corresponds to nucleotide position 4 and/or a G substitution at a position that corresponds to nucleotide position 122, wherein the nucleotide numbering is based on the nucleotide sequence of SEQ ID NO:1.

4. (canceled)

5. The polynucleotide of claim 1, wherein (a)-(g) are from the same AAV.

6. The polynucleotide of claim 1, wherein (a)-(g) are from different AAV.

7. The polynucleotide of claim 1, wherein said ITR further comprises non-AAV cis elements.

8. The polynucleotide of claim 7, wherein the non-AAV cis elements are selected from the group consisting of promoters, enhancers, chromatin attachment sequences, telomeric sequences, microRNAs, and combinations thereof.

9. The polynucleotide of claim 7, wherein the non-AAV cis elements do not comprise a promoter.

10. The polynucleotide of claim 1, further comprising one or more insulator sequence.

11. The polynucleotide of claim 1, further comprising a heterologous nucleotide sequence.

12. A viral vector comprising the polynucleotide of claim 1.

13. The viral vector of claim 12, which is an AAV vector.

14. A recombinant AAV particle comprising the polynucleotide of claim 1.

15. A chimeric AAV particle, comprising an ITR from any AAV serotype or the synthetic ITR of claim 1, wherein additional AAV cis elements are from a different AAV serotype than the ITR.

16. A method of transcribing a heterologous nucleotide sequence in a cell, comprising introducing into the cell the polynucleotide of claim 11.

17. (canceled)

18. A method of delivering a nucleic acid to a cell, comprising introducing into a cell the recombinant AAV particle of claim 14.

19. A method of producing a recombinant AAV particle, comprising providing to a cell permissive for AAV replication:
(a) a recombinant AAV template comprising (i) a heterologous nucleic acid, and (ii) the synthetic ITR of claim 1; and
(b) a polynucleotide comprising Rep coding sequences and Cap coding sequences;
under conditions sufficient for the replication and packaging of the recombinant AAV template;
whereby recombinant AAV particles are produced in the cell.

20. A method of producing a recombinant AAV particle, comprising providing to a cell permissive for AAV replication:
(a) a recombinant AAV template comprising (i) a heterologous nucleic acid, and (ii) a wildtype ITR from any AAV serotype or the synthetic ITR of claim 1; and
(b) a polynucleotide comprising Rep coding sequences and Cap coding sequences, wherein the Rep and Cap coding sequences are from a different AAV serotype;
under conditions sufficient for the replication and packaging of the recombinant AAV template;
whereby recombinant AAV particles are produced in the cell.

21-27. (canceled)

28. A method of administering a nucleic acid to a mammalian subject comprising administering to the mammalian subject a cell that has been contacted with the recombinant AAV particle of claim 14 under conditions sufficient for the AAV particle vector genome to enter the cell.

29. (canceled)

30. A method of administering a nucleic acid to a mammalian subject comprising administering to the mammalian subject the recombinant AAV particle of claim 14.

31-35. (canceled)

36. A method of enhancing promoter function of an adeno-associated virus (AAV) inverted terminal repeat (ITR) relative to a corresponding wildtype unmodified ITR, comprising modifying the ITR to produce the synthetic ITR of claim 1.

37. A method of reducing promoter function of an adeno-associated virus (AAV) inverted terminal repeat (ITR) relative to a corresponding wildtype unmodified ITR, comprising modifying the ITR to produce the synthetic ITR of claim 3.

38-41. (canceled)
